(19) United States
(12) Patent Application Publication
(10) Pub. No.: US 2009/0136918 A1
(43) Pub. Date: May 28, 2009

(54) QUANTIFICATION OF MICROSPHERE SUSPENSION HYBRIDIZATION AND USES THEREOF

(76) Inventor: Heather Newkirk, Overland Park, KS (US)

Correspondence Address: ERICKSON, KERNELL, DERUSSEAU & KLEYPAS, LLC, 800 W. 47TH STREET, SUITE 401, KANSAS CITY, MO, 64112, US

(21) Appl. No.: 11/465,108

(22) Filed: Aug. 16, 2006

Related U.S. Application Data
(60) Provisional application No. 60/708,734, filed on Aug. 16, 2005.

(51) Int. Cl. C12Q 1/68; G01N 33/00

(52) U.S. Cl. 435/6; 436/94

(57) ABSTRACT

A novel suspension hybridization assay was used to determine nucleic acid copy number by flow cytometry. The assay was validated with low copy (lc) products ranging in length from 100 to 2304 bp conjugated to spectrally-distinct polystyrene microspheres. In the example provided herein, these conjugated microspheres were used as multiplex hybridization probes to detect homologous sequences in genomic DNA extracted from cytogenetic cell pellets and labeled with biotin-dUTP. Hybridization was detected with phycoerythrin-labeled streptavidin and analyzed by flow cytometry. Copy number differences were distinguishable by comparing the mean fluorescence intensities of test probes with a diploid reference probe in genomic DNA of patient samples and abnormal cell lines. The assay is capable of distinguishing a single allele and three alleles at a test locus from a biallelic reference sequence, regardless of chromosomal context. The assay is an improvement on previous methods which require prior amplification of locus-specific target DNA because, lc probes provide adequate specificity and sensitivity for accurate copy number determination of homologous targets. Because of its high sensitivity and accuracy, the assay is useful for determination of nucleic acid copy number for a variety of applications, including determination of genomic copy number in humans, animal models of disease and in solution, measurement of transcript levels, forensic DNA analysis, and quality control analysis in agriculture.

### CROSS REFERENCE TO RELATED APPLICATION

This application claims the benefit of the prior filed, co-pending provisional application Ser. No. 60/708,734, filed Aug. 16, 2005, which is hereby incorporated by reference.

### SEQUENCE LISTING

A printed Sequence Listing, hereby incorporated by reference, accompanies this application, and has also been submitted with identical contents in the form of a computer-readable ASCII file on a floppy diskette.

### BACKGROUND OF THE INVENTION

1. Field of the Invention

The present invention concerns materials and methods for the detection of chromosomal abnormalities using low copy nucleic acid hybridization probes. More particularly, the present invention concerns quantification of chromosomal abnormalities in nucleic acid sequences through hybridization of microsphere-conjugated, low copy nucleic acid probes to labeled target nucleic acid. Still more particularly, the present invention concerns conjugating a spectrally-encoded microsphere to a low copy or single copy nucleic acid probe, hybridizing the probe to a fluorochrome-labeled target prepared directly from subject-derived nucleic acid, detecting the product of the hybridization reaction, and quantifying the detected response by comparing the detected response with the detected response generated by a similarly hybridized reference probe.

2. Description of the Prior Art

Diagnosis or predisposition to human diseases often depends on the quantification of specific nucleic acid sequences in the genome or transcriptome. Current methods for detecting numerical and structural chromosomal abnormalities include chromosomal banding, fluorescence in situ hybridization (FISH), array comparative genomic hybridization (aCGH) and other microarray techniques, multiplex amplifiable probe hybridization (MAPH), multiplex ligation-dependent hybridization (MLPA), Southern analysis, and quantitative polymerase chain reaction (qPCR). Quantification of gene expression by hybridization of cDNA is typically carried out by microarray analysis (Brown et al Science 270:467-70, 1995).

Numerical abnormalities are readily detectable by chromosomal banding but structural abnormalities such as deletions or duplications need to be large (˜3-5 Mb) and actually disrupt banding pattern in order to be reliably detected by chromosomal banding. FISH and Southern analyses require a large quantity of patient material, involve time consuming experimental procedures, and the level of detection achieved is ˜20 kb to 650 kb. The resolution of aCGH is limited to the size and density of cloned probes (˜40 kb for amplification of a sequence and 70-130 kb for deletions) (Shaffer and Bejjani, Hum. Reprod. Update, 10 (3):221-6 (2004)) and the variability of fluorescent signal can affect accuracy of interpretation (Oostlander et al., Clin. Genet., 66 (6):448-95 (2004)). MAPH has a resolution of approximately 50 kb (Armour et al., Nucleic Acid Res., 28 (2):605-09 (2000)) and while MLPA has higher resolution than other techniques, it is time consuming, and sensitive to single nucleotide polymorphisms at or near ligation sites which can lead to false negative results. Like qPCR, care must be taken to prevent contamination (Schouten et al., Nucleic Acid Res., 30 (12):e57 (2002)), and neither technique is easily multiplexed (ie. combining multiple probe sets in a single reaction).

Suspensions of spectrally-encoded polystyrene microspheres, coupled to synthetic DNA sequences, can be hybridized to a nucleic acid target sequence and detected by conventional flow cytometry as disclosed in U.S. Pat. Nos. 5,736,330, 5,981,180, and 6,057,107. These assays utilize 10-50 nucleotide oligonucleotide probes for hybridization to the target DNA. Other multiplexed data acquisition and analysis platforms for flow cytometric analysis of microsphere-based assays have been disclosed (Fulton et al., Clin. Chem., 43 (9):1749-56 (1997)). Standard nucleic acid hybridization assays are well known in the art and involve using a labeled nucleic acid probe to identify related target DNA or RNA molecules within a complex mixture of unlabeled nucleic acid molecules. Microsphere hybridization has been shown to be both accurate and sensitive for quantifying abundance of amplified ribosomal sequences from microorganisms in environmental samples (Spiro et al. Applied and Environmental Microbiology, 2000, p. 4258-4265, Vol. 66, No. 10). However, current assays require amplification of target DNA prior to hybridization with the probe, and current probes do not provide adequate specificity and sensitivity to accurately quantify genomic copy number directly from patient DNA samples. Polymerase chain reaction (PCR) amplification of the target DNA prior to assaying can take up to 5 hours to complete for each sequence detected, and when FISH analysis is used, the hybridization, detection and analysis process can take up to 2 days per probe per sample. Additionally, the requisite amplification step can complicate chromosomal copy number estimation of target sequences because the amplification is inherently logarithmic and difficult to control. However, previous attempts to directly analyze unamplified genomic DNA have been unsuccessful (Borucki et al., J. Clin. Microbiol., 43 (7):3255-59 (2005)).

The following prior art differs from the present invention, since in all methods below, prior amplification of target sample was required, and the probes coupled to microspheres were oligonucleotides which were not defined as single or low copy in all instances. Highly reiterated sequences in the genome or transcriptome do not necessarily require amplification for reliable detection, but single copy or low copy number sequences in large complex genomes do have this stipulation in order to obtain adequate sensitivity for detection. The following is a list of published and patented work involving microsphere suspension hybridization. The patents listed below are distinguished from the instant invention, since they are not enabling in terms of direct quantification of copy number of sequences in the nucleic acid target.
a. Spectrally encoded microsphere-conjugated analyte analysis: U.S. Pat. Nos. 6,649,414, 6,449,562, 6,395,470
b. Detection of PCR and DNA using oligonucleotides: U.S. Pat. Nos. 6,057,107, 6,060,240, 5,981,180, 5,736,330, 6,916,661, and 5,981,18
c. Microsphere suspension array hybridization technique involving hybridization of oligonucleotide sequences to PCR-amplified DNA: U.S. Pat. Nos. 5,736,330 & 6,057,107 and Fulton et al. Advanced multiplexed analysis with the FlowMetrix system, Clin Chem 1997 September; 43 (9): 1749-56.
d. U.S. Pat. No. 5,981,180 discloses a method of using color-coded beads in conjunction with flow cytometry to perform multiplexed biological assay
e. Method for multiplexed analysis of DNA fragments attached to microspheres and quantification of nucleic acid using photoactivatable label attached to the target nucleic acid was patented by Dattagupta and Nanibhushan, 2001; U.S. Pat. No. 6,620,586.
f. Patents pertaining to the attachment of oligonucleotides to microspheres, and detection of PCR products, polymorphism or methylation detection in hybridization assays follow. The following technologies may be used with that of the instant invention, but are distinct from it. “Nanoparticles having oligonucleotides attached thereto and uses therefor” U.S. Pat. Nos. 6,903,207, 6,902,895, 6,878,814, 6,861,221, 6,828,432, 6,818,753, 6,812,334, 6,777,186, 6,773,884, 6,767,702, 6,759,199, 6,750,016, 6,740,491, 6,730,269, 6,720,411, 6,720,147, 6,709,825, 6,682,895, 6,677,122, 6,673,548, 6,645,721, 6,610,491, 6,506,564, 6,495,324, 6,417,340, 6,361,944,
g. Other microsphere conjugated probe arrays for detection of particular oligonucleotide sequences: U.S. Pat. Nos. 6,890,741, 6,858,394, 6,919,206.
h. Mutation screening using PCR for target amplification: U.S. Pat. Nos. 6,528,261, 6,340,566
i. Mutation screening using primer extension: U.S. Pat. No. 6,537,748
j. Detection of pathogens using microspheres using amplified targets: U.S. Pat. Nos. 6,858,387, 6,620,586, 6,821,754, 6,821,519, 6,818,397

Accordingly, what is needed in the art are methods of rapidly determining the sequences of aneuploid or aneusomic domains using minute amounts of sample or patient material. What is further needed are methods for determining such sequences that are more accurate and do not require complex and time consuming steps of other current methods. What is still further needed are methods of determining numerical abnormalities of chromosomal or genomic nucleic acid sequences that do not require amplification of target DNA prior to hybridization with a probe.

### SUMMARY OF THE INVENTION

The present invention overcomes the problems outlined above and provides a novel method for identifying chromosomal abnormalities based on measurement of genomic copy number. It may also be used to determine DNA (or RNA) concentration of a solution or to determine levels of one or more transcripts in a complex mixture of nucleic acids. Generally speaking, the method of the present invention includes a microsphere suspension hybridization assay utilizing low copy genomic hybridization probes to determine copy number of a specific sequence relative to a reference sequence or standard curve. Sufficient accuracy is achieved to distinguish normal copy number which is generally two for autosomes from hemizygosity or from three or more alleles. This assay allows for the direct analysis of whole genomic DNA (or RNA) using flow cytometry and, if necessary, can follow routine cytogenetic analysis without requiring large patient sample quantities, additional blood draws, locus-specific amplifications or time-consuming genomic purification methods. It is notable therefore that copy number determination at a single locus can be carried out within a complex background of sequence consisting of the complete genome. This exquisite level of discrimination can also be used to determine copy number of rare transcripts against the background of the complete transcriptome, or for detection of extremely dilute or low concentrations of specific nucleic acid sequences within heterogeneous solutions of nucleic acids.

Unlike current hybridization assays with oligonucleotide probes, prior amplification of locus-specific target DNA is not required because the longer low copy hybridization probes of the present invention provide the increased specificity required for direct detection of homologous, unique genomic target sequences. For example, the present invention can be used to detect gains or losses in copy number in patient genomic samples with less than 90 minutes, more preferably less than one hour, even more preferably, less than 50 minutes, more preferably less than 45 minutes, still more preferably less than 40 minutes, even more preferably less than 35 minutes and still more preferably less than about 30 minutes of hands-on laboratory time, regardless of the number of probes present in each reaction per sample. Additionally, one can combine multiple independent low copy probe-conjugated microsphere sets in the same hybridization assay to independently measure multiple copy number changes. Multiplexed analysis not only decreases the amount of sample required per assay, as compared to FISH, but also decreases the analysis time and overall cost per sample tested.

In preferred forms, the present invention utilizes low or single copy hybridization probes specially designed to hybridize to a unique locus in the haploid genome sequence with high specificity. The method for probe selection and synthesis is disclosed in U.S. Pat. No. 6,828,097 and in pending U.S. application Ser. No. 09/854,867 (U.S. Patent Publication No. 2005/0064449), the teachings and content of which are hereby incorporated by reference. As can be appreciated, these initial steps require knowledge of the sequences of both the target and genomic repeats, information that is increasingly available owing to the Human Genome Project and related bioinformatic studies. Furthermore, readily available computer software is used to derive the necessary low copy sequences.

Preferably, at least two different probe sequences are selected and synthesized. At least one set of probes should be selected and synthesized for recognition of a particular nucleic acid sequence wherein the abnormality, if present, would reside (test probe), and another set of probes should be selected and synthesized for recognition of a reference sequence (reference probe). The low copy probes should be at least about 60 base pairs and generally no more than 2500 base pairs in size. Preferably, the probes are between 60 and 1000 base pairs, more preferably between about 80-500 base pairs and most preferably between about 90-110 base pairs. It was found that microspheres conjugated to low copy probes of about 100 base pairs produced well-defined mean fluorescence distributions and consistently higher secondary fluorochrome mean fluorescence intensity values, and thus more precisely reflected the actual copy numbers. The shorter probe conjugates are also more stable and can be used in hybridization reactions for more than two months after conjugation when properly stored, preferably in the dark and at about 4° C. This results in less lot-to-lot variation in labeled microsphere stocks, thereby reducing the effort required to conjugate, quantify and qualify probe-conjugated microspheres. Longer conjugated probes showed degraded hybridization efficiency within two weeks after conjugation.

During synthesis, the probes can be amine-tagged, depending upon the particular conjugation reaction, for conjugation to spectrally-distinct microspheres. Preferably, the probes are conjugated to the microspheres via a modified carbodiimide reaction (as described in Example 1) wherein the microspheres have been carboxylated. Other methods for coupling nucleic acids to microspheres have been developed that are equivalent in scope to the instant method. Another common method for conjugating DNA to microspheres binds strepatividin-coated beads to probes containing a modified nucleotide with a biotin moiety at the 5′ terminus. Gold nanoparticles have been conjugated to thiol-modified nucleic acids in U.S. Pat. No. 6,361,944. Protein-nucleic acids have been conjugated to beads in U.S. Pat. Nos. 6,468,546, 6,838,243, 6,833,246, 6,828,146, 6,828,142. Conjugation of oligonucleotides to microspheres has also been carried out via an electrophilic tether, namely N-chloroacetamidohexyl phosphoramidite reagent (Guzaev, et al Bioorg Med Chem. Lett. 1998 Dec. 15; 8 (24):3671-6). DNA has been also conjugated to semiconductor nanocrystalline particles (Taylor, J. R., Fang, M. M., & Nie, S. M. Probing specific sequences on single DNA molecules with bioconjugated fluorescent nanoparticles. Anal. Chem. 72, 1979-1986, 2000; W. Z. Guo, J. J. Li, Y. A. Wang, X. G. Peng, Conjugation chemistry and bioapplications of semiconductor box nanocrystals prepared via dendrimer bridging. Chem. Mater. 15, 3125-3133 (2003), and U.S. Pat. No. 6,630,307). In a preferred embodiment, the microspheres are internally dyed, fluorescent, polystyrene beads with various spectral addresses, and are conjugated to low copy probes such that various combinations of low copy probe-conjugated beads result, wherein the reference probes are designated with a distinct spectral address. The different spectral addresses are recognized by the flow cytometer and allow for multiplexed reactions with various low copy probes attached to different microsphere sets.

The target nucleic acid sequence is then prepared for hybridization. Unlike other methods, the target sequence is not pre-selected or amplified, such that in the present invention an entire copy of a genome (or transcriptome) can be hybridized for analysis. Depending on the source and condition of the sample, the DNA (or RNA) is extracted from the cells and, if necessary, can be replicated in vitro using any conventional method. Preferably, the nucleic acid is replicated in vitro using a GenomiPhi kit (Qiagen, Valencia Calif.), which utilizes less than one ng of sample nucleic acid and requires less than 20 minutes hands-on time. The DNA is then labeled by any conventional means, preferably by a direct labeling step during in vitro replication or by an indirect labeling system consisting of a label and a reporter molecule that has an affinity for that label. The nucleic acid target is labeled with an identifying label such as a fluorophore, an enzymatic conjugate, or one selected from the group consisting of biotin or the moieties recognized by avidin, strepavidin, or specific antibodies. There are several types of non-isotopic identifying labels. One type is a label which is chemically bound to the nucleic acid target and serves as the means for direct identification. An example of this would be a fluorochrome moiety, which upon application of radiation of proper wavelengths will become excited into a high energy state and emit fluorescent light. Directly labeled fluorescent nucleotides such as Cy3-dUTP are known in the art and would be suitable forms of labeling of target DNA for use with the instant invention. Other methods of direct labeling of DNA would also be suitable (an example would be the amino-allyl labeling marketed as the Ulysses method (Kreatech, Netherlands), however in such instances the genome DNA would have to be fragmented (by DNAse I, shearing, or other enzymatic digestion) to a suitable size for hybridization prior to addition of the labeled target to probe conjugated microspheres. In a preferred embodiment, the nucleic acid target is preferably labeled by nick translation using a modified or directly labeled nucleotide (Rigby et al., J. Mol. Biol., 113:237-251, 1977) in the conventional manner using a reactant comprising the identifying label of choice (but not limited to) conjugated to a nucleotide such as dUTP or dATP. The fragments are either directly labeled with fluorophore-tagged nucleotide or indirectly labeled by binding the labeled duplex to a fluorescently-labeled antibody that recognizes the modified nucleotide that is incorporated into the fragment as described below. Nick translations (100 μL) utilize endonuclease-free DNA polymerase I (Roche Molecular Biochemicals and DNase I (Worthington Chemical). Each fragment is combined with DNA polymerase I (4 units/microgram DNA), DNase (0.01-3 microgram/100 μL reaction), labeled nucleotide (0.05 mm final) and nick translation buffer. The reaction is performed at 15° C. for 60 minutes and yields a variety of labeled probe fragments of different nucleotide sizes in ˜300 to 1000 bp size range. Alternatively, biotin-dUTP or digoxygenin-dUTP can be incorporated during the in vitro replication procedure and resulting labeled sample can be treated with DNAse I or sheared by some other method to yield fragments ˜300 bp to 1 kb in length. Other methods for labeling and detecting nucleic acids in common use may be applied to the detection of low copy DNA conjugated microspheres of the present method. These include fluorochrome labels and fluorescent compositions such as energy transfer groups, conjugated proteins, antibodies, or antigens.

The conditions for the hybridization reaction will depend upon the particular nucleotide composition and the length of each low copy probe, and are easily determined by those of skill in the art. For hybridization, the sample sequence is diluted in a hybridization buffer solution containing the low copy probe-conjugated microspheres. The amount of probe-conjugated microspheres to be utilized will depend upon the amount of sample tested. Preferably, about 5 pg to 1 μg of sample, more preferably about 25-100 ng of sample, still more preferably about 30-70 ng, yet more preferably about 40-60 ng of sample, and most preferably about 50 ng of sample, is analyzed per hybridization reaction. Accordingly, the buffer solution preferably contains about 2,000-10,000 probe-conjugated microspheres, more preferably 2,000-6,000 probe-conjugated microspheres, still more preferably about 4,500-5,500 probe-conjugated microspheres, and most preferably about 5,000 probe-conjugated microspheres for each set to be hybridized. Once diluted, the hybridization reaction is heat denatured, preferably at about 95° C. and then hybridized overnight at a suitable hybridization temperature, preferably, at about 45 to 51° C. depending upon the probe nucleotide composition and length. The hybridized microspheres are then washed and centrifuged to remove unhybridized target sequence.

The supernatant is removed and the hybridized sample is stained or labeled with an amount of a modified reporter molecule or other suitable label, preferably one which acts as a secondary fluorochrome, to detect the labeled sample hybridized to the low copy probe-conjugated microspheres. The preferred reporter molecules are phycoerythrin-labeled streptavidin or anti-digoxigenin fluoroscein, which detect and bind the preferred target sequence labels, biotin and digoxigenin, respectively. The hybridized and labeled/stained sample is incubated at the same temperature used for the hybridization reaction, for a period of time sufficient for the reporter molecule to detect and bind the labeled target sequence. Afterwards, the sample is washed to remove residual stain. The samples are centrifuged, the supernatant removed and the stained hybridized microspheres are resuspended in an amount of hybridization buffer.

Before analysis by flow cytometry, the hybridized samples can be diluted depending upon the flow cytometer manufacturer's instructions. Preferably, about 2,000-6,000 microspheres of each set are analyzed per reaction, more preferably about 4,500-5,500 microspheres, and most preferably about 5,000 microspheres per set are analyzed per reaction. However, the amount of sample to be analyzed may depend upon the particular flow cytometer utilized for analysis. It will be appreciated that calibration and operating settings for the flow cytometer can be modified in a number of ways without undue experimentation, by those skilled in the art, to determine the optimal ranges for measuring a particular hybridization assay. These parameters will also depend upon the software employed for analysis. Fluorescent bead standards are widely available and can be used to calibrate the intensity of different fluorochrome detection channels of the flow cytometer. The instrument can also be calibrated with fluorescent reference standards based on surface-labeled beads calibrated in molecules of equivalent soluble fluorochrome (MESF) units. Photomultiplier tube voltage settings and thresholds for forward scatter, side scatter, flow rate, and various detection channels should preferably be optimized to minimize differences between fluorescence intensities of two different probes hybridized to a single patient sample with a normal genotype. Non-optimal voltage parameters are readily apparent and result in broad fluorescence peaks or non-linear data, whereas optimal parameters preferably result in tightly clustered microspheres with different spectral addresses when visualized using a side scatter plot. Preferably, these settings are determined from derived fluorescence measurements of arithmetic mean, geometric mean, median and peak channel.

It was found that most of the low copy probes of the present invention do not require significant optimization for composition, because probe quality depends mainly on the low copy nature of the sequence used to design it. Aside from methods of low copy probe design described in U.S. Pat. No. 6,828,097, reference and test probe pairs were optimized for equivalent thermal stability by matching sequences of similar length, and if possible, composition (in particular, the percentage of G and C nucleotides) in an effort to mitigate secondary structure formation at or near the hybridization temperature of each probe. Sequences that are paralogous to other genomic loci produce fluorescence intensity ratios that exceed established confidence intervals for known genotypes. The present invention overcomes this limitation by use of low copy probes that are strictly not homologous to other genomic segments.

The hybridized samples are then analyzed by flow cytometry, preferably using dual laser detection, whereby the cytometer co-selects for the spectral addresses of the microspheres and the secondary fluorochrome bound to the sample sequences in order to identify and quantify the hybridized probes. Copy number differences are distinguishable by comparing the mean fluorescence intensities of the test probes with the intensities of the disomic reference probe. Specifically, the signal of each sample sequence, hybridized to its complementary probe-conjugated microsphere is determined by quantifying the fluorescence intensity of the secondary fluorochrome attached to the sample sequence. For example, the reference probe serves as an internal reference by which one copy (deletion) can be distinguished from two (normal), and two copies can be distinguished from three (duplication) based upon differences in fluorescence intensity. Compatible microsphere spectral addresses are selected to minimize overlap with the emission wavelengths of any unbound secondary fluorochrome (reporter molecule). This can be confirmed by comparison with results obtained from otherwise identical unconjugated and unhybridized microspheres. A negative control may also be maintained using a reaction tube containing all of the components except for the sample nucleic acid in order to determine background fluorescence in the secondary fluorochrome detection channel. Preferably, the system is flushed with distilled water between runs to remove any residual microspheres.

Preferably, geometric mean or median fluorescence ratio is used to measure copy number in the present hybridization assay because it provides the smallest confidence intervals (95%) and residuals for all of the genotypes with no overlap between genotypes. In sequences containing a deletion, the expected value for the geometric mean or median fluorescence ratio of test probe to reference probe intensity is about 0.5, which reflects a genomic copy number difference of one versus two. In sequences containing a duplication (or insertion), the ratio is expected to be about 1.5, which reflects a genomic copy number difference of three versus two. Ratios greater than 1.7 typically indicate increases in copy number of up to five additional copies of a sequence. And in normal sequences the ratio is expected to be about 1.0, which reflects the normal diploid copy number for both genotypes. In practice of the present invention, deviation from these expected ratios is preferably minimized, such that ratios are within a confidence interval of about 80%, more preferably about 85%, still more preferably within about 90%. In the preferred embodiment, these ratios should not be less than the 95% confidence interval and there should be no overlap between genotypes, thus allowing for a more accurate determination of copy number. Preferably ratios reflecting a deletion should be about 0.25-0.75, more preferably about 0.40-0.60, even more preferably between about 0.45-0.55, and most preferably about 0.49-0.51. Ratios reflecting either insertions or duplications should preferably be about 1.25-1.75, more preferably about 1.40-1.60, still more preferably between about 1.45-1.55, and most preferably between about 1.49-1.51. Ratios reflecting normal genotypes should preferably be about 0.75-1.25, more preferably about 0.90-1.10, still more preferably between about 0.95-1.05, and most preferably between about 0.99-1.01.

It was found that the present invention consistently distinguishes copy number differences between a diploid reference sequence and a chromosomal deletion (single allele), insertion, trisomy or duplication (three alleles), as well as increases in copy number up to five additional copies (five alleles). This assay can detect very low copy or rare sequences, even if the particular test sequence is only present in 1 copy per haploid genome. The extent of the nucleic acid rearrangement can be defined within 62 bp, which is significantly more precise than array CGH, Southern analysis and comparable to the precision of MLPA. The microsphere bead hybridization assay of the present invention improves resolution, increases signal to noise ratio, and is easily amenable to multiplexing, making it a high throughput approach. The ability to multiplex probes conserves available sample or patient material and decreases time needed to make diagnoses. Further, contamination of heterologous genomic sequences was shown to not impact the hybridization efficiency of the assay. Consequently, the microsphere hybridization platform may be more appropriate in some diagnostic situations instead of FISH, array CGH (aCGH), expression or tiling microarray, Southern analysis, multiplex amplifiable probe hybridization (MAPH), multiplex ligation probe hybridization (MLPA) and quantitative PCR (qPCR).

This assay is easily amenable to high throughput applications. For example, with genomic arrays of low copy microsphere-conjugated suspension hybridization probes, it should be feasible to rapidly determine and/or define abnormal chromosomal sequences based on differences in copy number relative to one or more conserved reference loci whose copy number is essentially invariant among different individuals. Hybridization of a dense set of low copy probes that span a chromosomal region could be used to initially screen for the boundaries of aneusomic domains. Subsequent FISH studies could then provide a chromosomal context for these genetic alterations. One can envision high throughput applications, because approximately 3840 different products could be assayed on a single high-density microtiter plate using the current set of 10 spectrally distinct encoded beads in each well. With available flow cytometers, assuming a minimum probe resolution of 20 kb, a single plate could provide high-resolution copy number determination for a 20 Mb domain within 90 minutes of data acquisition time.

Because this invention is both more sensitive and more accurate than other art-related technologies, it may be used for other diagnostic applications (in addition to those presented in Examples 1 and 2, given herein), which have previously required either highly reiterated probes or with probes requiring prior target amplification.

One aspect of the present invention provides for detection of chromosomal imbalances. One of the most significant implications of detection of patient deletions (and possibly triallelic inheritance as well) is the resulting imbalance of alleles observed. That is, while haploinsufficiency or over expression of a transcribed genomic sequence may contribute to disease, the presence of a single mutant allele that is unmasked by a deletion of the wild type allele (or a 2:1 ratio of mutant to wild type alleles) may be as or a more important determinant of some abnormal clinical phenotypes. Judicious selection of low copy intervals may not only detect these chromosome region imbalances, but will also enable detection of mutations or SNPs that are found within these domains. Due to the efforts of the haplotype mapping consortium, detailed genomic locations and population frequencies of millions of SNPs are now available. Based on the work of some of the inventors of the present application, some of these are clearly associated with various congenital disorders.

The lc (low copy) probes used to detect chromosome imbalances using this microsphere assay can be selected based on the prior knowledge of the locations of published polymorphic sequences or mutations. After a chromosomal abnormality is detected using the assay, the hybridized microsphere is then recovered, the patient-derived sequence melted away from the conjugated sequence, the beads centrifuged down and the supernatant containing the melted strand then used in the subsequent step. This sequence could then be directly analyzed, ie. with a fluorescent PCR assay such as Taqman (Applied Biosystems) or with molecular beacon assays, or amplified and sequenced directly to determine the genotype of the hybridized allele(s). Duplexes comprised of patient samples annealed to conjugated lc probes can be trimmed to blunt ends using a nuclease (eg. S1 or Exonuclease I), then treated with Cleavase (Third Wave Technologies) to digest the DNA at mismatched sites, and the fragments separated by either dHPLC (using a Wave System, Transgenomic) or electrophoresis. Two shorter fragments whose lengths summed to the probe length would be expected (this would require conjugation of the bead to a lc probe derived from a known homozygote for the SNP—rather than from a mixture of genomic DNAs, which is the current source of template for probe synthesis).

Another alternative genotyping procedure could also use microsphere hybridization in which the recovered fragments would be used as targets in subsequent microsphere hybridization assays. The carboxylated microspheres are coupled to 5′ amino labeled probes (50-70 nucleotides) in which the SNP is situated in the middle of the sequence. Since the sequence of these probes is determined by the location of the SNP, their sequences are not necessarily low or single copy. Two distinct microspheres are employed, one coupled to the wild type sequence (reference probe), and the other containing the variant SNP sequence (test probe). Hybridization is carried out as described previously, except the hybridization time is reduced to 15 to 30 minutes due to the reduced complexity of the target sequence. The probe containing the base mismatch is destabilized by stringent washes following the hybridization and dissociates. The microsphere containing an exact sequence match will produce much stronger mean fluorescence intensity. U.S. Pat. No. 6,355,431 and related patents disclose that microsphere-based hybridization can be used to initiate genotyping, however, these patents anticipate that the DNA captured by hybridization was previously obtained by target amplification. The instant invention eliminates the requirement for target amplification prior to hybridization.

At least in the case of deletions, the haplotypes of tightly linked SNPs (the phase) in the same labeled nucleic acid could be determined with this assay. The novelty here is that the mutation(s) and the deletion are effectively detected in a single assay.

Another aspect of the present invention could be utilized in livestock testing. Duplicated or deleted regions can be associated with Quantitative Trait Loci (QTLs) (Montaldo et al. Use of molecular markers and major genes in the genetic improvement of livestock. Animal Biotechnology Volume 1. No 2. Aug. 15, 1998, 83-89), which are currently assayed by methods such as PCR and qPCR assays for cattle and sheep. Because of its high-throughput nature, the microsphere assay for QTL deletion or duplication markers using the instant invention is more suitable for use with large herds than qPCR or PCR tests. Test probes specific to a series of QTL loci can be conjugated to spectrally distinct microspheres and hybridization efficiencies of these probes can be compared to a standard reference probe, even HOXB1 in the case of livestock species, in an effort to discern ratios above or below 1 indicating duplications or deletions of QTLs, respectively. Analysis methods are in accordance with those described above.

In another aspect of the present invention, transgenic or knockout mice (or other organisms) could be tested for successful insertion/deletion of sequences quickly in a transgenics core using the instant invention. Current methods involve PCR or qPCR, such as those described in Nature Genetics 21, 249-251, Modeling cancer in the mouse (1999). Test probes specific to the transgenic or knocked out sequence (test probes) can be conjugated to spectrally distinct microspheres as well as a sequence in the genome adjacent to the transgenic or knocked out sequence (reference probe). Genomic DNA or complementary DNA from the transgenic or knock-out animal is then extracted and labeled as described earlier. Hybridization and detection of test and reference probes is also as previously noted. If the MFI ratio of the test to reference probe is greater than 1, then the test probe sequence has been successfully inserted into the transgenic animal. If the MFI ratio is 0.5, then one copy of the test probe sequence has been successfully knocked out or deleted from the animal genome. If the test probe fails to hybridize, this would indicate that both copies of the test probe sequence have been deleted from the knock-out animal tested.

Another aspect of the present invention provides for the detection of levels of genetically modified crops. PCR based methods have become one of the standard approaches for quantifying genetically modified organism (GMO) content in crops (for example see: Zimmermann A, Hemmer W., Liniger M., Luthy J., Pauli U. Lebensmittel-Wissenschaft und-Technologie, 31 (7): 664-667, 1998; Studer E., Rhyner C., Lüthy J., Hübner P. Zeitschrift für Lebensmitteluntersuchung und-Forschung A. 207 (3): 207-213, 1998; Holst-Jensen, Sissel B. Running, Astrid Løvseth, Knut G. Berdal. Analytical and Bioanalytical Chemistry. Volume 375, Number 8, 985-993, 2003), however these methods have some limitations. They are particularly susceptible to false positive results with low levels of contamination and at the limits of sensitivity (<2%), these methods can give variable results including false negative findings (Ahmed F., Trends in Biotech. 20:215-223, 2002). The existing qPCR assays that the USDA uses are particularly variable at the 1% threshold of detection. A commercial improvement in the reliability of GMO assays at the threshold would be valuable since the European Union set this value as their upper limit for agricultural imports. A test probe specific to the particular genetic modification can be conjugated to a spectrally distinct microsphere. A reference probe of known copy number in the particular crop genome being investigated would also be conjugated to a spectrally distinct microsphere. All experimental procedures would be as described previously. A MFI ratio of test to reference probe of 0.5 would indicate that the genetically inserted organism has inserted in one copy, a ratio of 1 would indicate two copies, and a ratio of 1.5 or greater would indicate three or more copies of the GMO. If the test probe fails to hybridize, this could indicate the absence of the GMO in the crop genome.

Using the methods of the present invention, the sensitivities achieved in quantification of low copy sequences in human genomic DNA are considerably better than those currently obtained using PCR for low level detection of GMO content. Furthermore the methods of the instant invention are not susceptible to false positive detection due to low level contamination, since amplification of target sequences is unnecessary. Based on the ability to discriminate low level copy number differences demonstrated with the instant invention, one of skill in the art would appreciate that the microsphere suspension hybridization assay would be a superior test for GMO contamination.

Another aspect of the present invention provides methods for measuring transcript levels in cDNA or in libraries relative to standard transcript equivalents or to an internal housekeeping reference gene. Current methods to detect the presence of specific transcripts in cDNA or in libraries are typically tested using qPCR (Physiol Genomics. 2003; 16 (1):90-8), or PCR (Development. 1992; 116 (3):555-61), or by microsphere hybridization (JS Patent 6,875,568). Current methods of PCR and microsphere hybridization do not quantify transcript levels, and only detect the presence or absence of transcript. The instant invention can enable the detection and quantification of low copy or rare mRNA sequences (˜5 copies per hybridization reaction) in cDNA. (Note: 5 copies was the number experimentally determined in Example 1 during the detection of sequences in a heterologous genomic environment.) Methodology used is in accordance with the methods described above.

Another aspect of the present invention provides methods for measuring changes in transcript levels in response to stimulus (environmental or chemical perturbation) or over time. Current methods are based namely on PCR (J. Immunol. 2001. Mar. 15: 166 (6):3663-71), or RTPCR (Biomaterials. 2003 July; 24 (15):2561-73). A probe specific to the transcript of interest can be conjugated to microspheres and its fluorescence intensity compared to a transcript of known expression level, such as a housekeeping gene. If the transcript level increases after the stimulus, then the MFI ratio of the test to reference probe would be greater than 1. If the transcript level decreases then the ratio would be less than 1. Standard protocols would be used for the hybridization and detection.

Another aspect of the present invention provides methods for detecting chimerism or mosaicism within a mixed population of cells in a sample. Chimerism is defined as an individual or tissue sample composed of cells derived from two genetically distinct zygotes. Mosaicism is defined as an individual or tissue sample with at least 2 or more cell lines differing in genotype or chromosomal complement derived from a single zygote. For example, a X or Y chromosome specific probe from the nonpseudoautosomal region can be used to detect chimerism, mosaicism consisting of cells from two differentiated cell lines of opposite sex (eg. XX vs. XY) or of differing sex chromosome complement (i.e 45,X vs. 46,XX). These probes would demonstrate difference in copy number compared to a reference cell line which is nonmosaic or nonchimeric. Using an autosomal reference probe, for example, a 45,X individual will show a mean fluorescence ratio of ˜0.5 and a 46,XX individual will have a ratio of ˜1.0. Individuals with mosaic or chimeric constitution will have a ratio of between 0.5 and 1.0 if the chimera is of opposite sex or the mosaic is of differing sex chromosome complements. Examples of chimerism in humans include exchange of hematopoictic stem cells by dizygotic twins in utero; fusion of two zygotes into one individual in utero; and tissue or organ transplantation. Chimerism is currently detected by methods such as interphase FISH and cytogenetics in the case of sex-mismatched cell transplants in certain cancers (Bernasconi P, Cavigliano P M, Genini E, Alessandrino E P, Colombo A, Klersy C, Malcovati L, Biaggi G, Martinelli G, Calatroni S, Caresana M, Boni M, Astori C, Bernasconi C. Leukemia. 1997 November; 11 (11):1989-90; Smith A, Robson L G, Sharma P, Shaw P J. Pathology. 1999 February; 31 (1):25-8; Ng I O, Chan K L, Shek W H, Lee J M, Fong D Y, Lo C M, Fan S T. Hepatology. 2003 October; 38 (4):989-98) or resulting from pregnancy (O'Donoghue K, Chan J, de la Fuente J, Kennea N, Sandison A, Anderson J R, Roberts I A, Fisk N M. Lancet. 2004 Jul. 10-16; 364 (9429):179-82) and by DNA polymorphism studies such as microsatellite detection in single cells (Yu N, Kruskall M S, Yunis J J, Knoll J H, Uhl L, Alosco S, Ohashi M, Clavijo O, Husain Z, Yunis E J, Yunis J J, Yunis E J. N Engl J Med. 2002 May 16; 346 (20):1545-52). Mosaicism is frequently detected by methods such as FISH and cytogenetics, but it is acceptable to detect this genotype with other DNA based methods (Lomax et al., Hum Genet. 1994 March; 93 (3):243-7; Krishnamoorthy et al, J Perinatol. 1995 January-February; 15 (1):47-50; Chen et al., Prenat Diagn. 2004 October; 24 (10):767-73).

In another aspect of the present invention, quantification of viral or bacterial load in tissues or plasma by comparison with a reference sequence is possible. Current methods only detect the presence or absence of such bacterial or vial contaminants by ELISA, PCR, or qPCR (Comp Med 2001 October: 51 (5): 406-12. Evaluation of diagnostic methods for Helicobacter bills infection in laboratory mice, Hodzic, E et al., Proc Natl Acad Sci USA. 2001 Nov. 20; 98 (24):13687-92; J Clin Microbiol. 2005 February; 43 (2):716-20; Arch Dermatol Res. 2005 February; 296 (8):345-52. Epub 2005 Jan. 4; J Virol Methods. Jul. 7, 2005). One method for pathogen detection does involve suspension array (J Microbiol Methods. 2005 Jun. 24, and US Pats 6,821,754, 6,821,519, 6,818,397, 6,790,611, 6,709,812, 6,632,607, 6,627,198), however in each of these cases, PCR amplification of the specific pathogens is required prior to hybridization. Using suspension array analysis, test probes specific to the virus or bacterium of interest can be conjugated to microspheres and tested with a reference probe, such as HOXB1.

Yet another aspect of the present invention provides for directly measuring concentrations of nucleic acids, including nucleic acids other than DNA, in solution by hybridization and comparison to sample at a known concentration using the above-described methodologies. Some current methods for RNA concentration measurement rely on hybridization to beads attached to solid surface support (Genome Research 14:2347-2356, 2004), PCR, and qPCR. Patented microsphere suspension array methods designed to quantitate nucleic acids bound involve prior amplification of products (US Pats: 6,620,586, 6,812,005, 6,355,431 and 6,858,387).

Another aspect of the present invention permits determination of differences in copy number at polymorphic genomic loci to establish relatedness for forensic or genetic predisposition studies. Instability of segmental duplication of genomic sequences is common in the human genome. This phenomenon can lead to diseases (usually constitutional or congenital) that result in haploinsufficiency or increased copy number of genes within the regions flanked by these duplications. The CMT1A example of the present invention was one of the earliest known examples of copy number instability leading to disease.

Some unstable segmental duplications are not strictly pathogenic. A subset of copy number differences among individuals (n=119) are associated with normal variation or polymorphism in the genome (Segmental duplications and copy-number variation in the human genome: Sharp A J, Locke D P, McGrath S D, Cheng Z, Bailey J A, Vallente R U, Pertz L M, Clark R A, Schwartz S, Segraves R, Oseroff V V, Albertson D G, Pinkel D, Eichler E E. Am J Hum Genet. 2005 July; 77 (1):78-88. 2005). Copy variation at these polymorphic loci are not confined to a single population, suggesting that they either are recurrent genetic events, having occurred separately in multiple founders, or were present prior to the human migration from Africa.

By appropriate selection of genomic probes from segmentally duplicated intervals, the instant invention can determine how copy number genotype varies among individuals. A DNA fingerprint of an individual can be obtained by carrying out copy number determination with a sufficiently large number of such probes conjugated to spectrally distinct microspheres. The copy number polymorphism frequency for these probes is relatively high, suggesting that these genomic markers will be sufficiently informative for forensic applications of genetic identity.

These same segmental duplications represent chromosomal architectures that predispose some individuals to undergo gametic rearrangement during meiosis. Although these individuals themselves are unaffected, they would be at higher risk for offspring with segmental aneusomy in regions with predisposing chromosome configurations. Other chromosomal rearrangements, such as reciprocal, Robertsonian and jumping translocations, inversions, isochromosomes and small marker chromosomes, may be susceptible to rearrangement related to genome structure or architecture. Segmental duplicons, A T-rich palindromes (Gotter A L, Shaikh T H, Budarf M L, Rhodes C H, Emanuel B S. Hum Mol. Genet. 2004 Jan. 1; 13 (1):103-15) and pericentromeric repeats have been localized to such rearrangement breakpoints. Analysis of the products of recombination at the junctions of the rearrangements has revealed both homologous recombination and non-homologous end joining (Shaw C J and Lupski J R, Hum Mol. Genet. 2004 Apr. 1; 13 Spec No 1:R57-64). By appropriate selection of probes within or adjacent to segmental duplicated regions of the chromosome, it should be feasible to determine which individuals are at increased risk for gametic chromosome rearrangements that would concomitantly increase the risk of abnormal offspring.

Further, genomic rearrangements which do not involve a loss or gain in copy number, such as inversions or translocations, can be assessed using the described method. A probe specific to the 5′ or 3′ terminus of the inverted or translocated sequence is conjugated to microspheres and hybridized to labeled genomic DNA containing the chromosomal abnormality (eg. ABL in the case of an ABL/BCR translocation causing chronic myelogenous leukemia). Hybridized microspheres are then recovered, the patient-derived sequence melted away from the conjugated sequence, the beads centrifuged down and the supernatant containing the melted strand then used in the subsequent step. The recovered fragments are then used as targets in subsequent microsphere hybridization assays using a test probe specific to the sequence bordering but not implicated in the inversion or translocation (eg. BCR on chromosome 22). Hybridization and detection may be carried out as described previously, except the hybridization reaction will involve the new test probe (BCR) as well as the test probe from the previous hybridization (ABL). If the MFI ratio of the two test probes is approximately equal to 1, this would indicate that the translocation or inversion has successfully been identified. If the second test probe fails to hybridize, then no abnormality was detected. The MFI ratio of the second test probe compared to the first test probe should not have a ratio greater than 1. No genomic assay in the prior art utilizing suspension array can detect the presence of translocations or inversions.

Thus, one preferred embodiment of the present invention provides a method of quantifying the copy number of a target nucleic acid sequence in a genome. The method generally comprises the steps of extracting nucleic acid containing said target nucleic acid sequence from a subject, attaching a label to said target nucleic acid, preparing a particulate-conjugated, low-copy, nucleic acid probe selected to complement said target nucleic acid sequence, hybridizing said probe to said target nucleic acid to form a hybridization reaction product, identifying said product through detection of said particulate, and quantifying said copy number of said target nucleic acid sequence through detection of said label on said product. Preferably, said particulate comprises a nanocrystalline particle, still more preferably, said particulate comprises a nanosphere, even more preferably, said particulate comprises a microsphere, and most preferably, said particulate comprises a spectrally-distinct polymer microsphere. In a particularly preferred embodiment, said microsphere comprises polystyrene, and even more particularly, said particulate comprises an internally-dyed, fluorescent, polystyrene bead having a determined spectral address. In some applications, the method further comprises the step of binding a fluorochrome to a biological moiety on said microsphere surface. In other preferred applications, the general method also includes a step selected from the group consisting of conjugating said probes to said microsphere via modified carbodiimide reaction wherein said microsphere has been carboxylated and conjugating said microsphere to said probe using an electrophilic tether, wherein said tether comprises N-chloroacetamidohexyl phosphoramidite. The extracted target nucleic acid comprises DNA selected from the group consisting of genomic DNA and complementary DNA. Labeling can be done by any conventional method, including via nick translation with an identifying label, directly labeled during an in vitro nucleic acid replication reaction, end labeling, and random priming. Labels can be any conventional labels, including fluorophores, enzymatic conjugates, fluorophore-tagged nucleotides, fluorescently-labeled antibodies bound to antigen-bearing nucleotides, biotin-dUTP, digoxygenin-dUTP, and combinations thereof. Preferably, the nucleic acid sequence of said probe is complementary to a low copy or single copy sequence in said subject's genome. Typical response ratios of target nucleic acid sequences containing a deletion of one or more base pairs is from about 0.1 to about 0.75 in comparison to a reference probe having a normal complement of said target nucleic acid sequence. Typical response ratios of target nucleic acid sequences containing an insertion or duplication of one or more base pairs is from about 1.25 to about 1.65 in comparison to a reference probe having a normal complement of said target nucleic acid sequence. Advantageously, the method can be adapted such that multiple probes having distinct nucleic acid sequences are conjugated to particulates having one or more distinct spectral addresses and hybridized to target nucleic acids. In preferred embodiments, said particulate comprises a streptavidin-coated or carboxylated bead bound to a biotin or amino moiety at the 3′ or 5′ terminus of said nucleic acid probe and has a diameter between 50 nm and 1 μm. Additionally, the amount of target sequence detected per 1 μg of heterologous genomic nucleic acid sequence is within the range of 50 pg to 5 ng and differences in copy number are detected with a genomic resolution of as little as 60 bp. Preferably, said quantifying step includes the step of detecting the spectral address of said product using flow cytometry. Preferred probes have a length of between 50 and 2500 bases, more preferably between 70 and about 2500 bases, still more preferably between 60 and about 1000 bases, even more preferably between 80 and about 800 bases, still more preferably between 90 and about 110 bases, and most preferably about 100 bases.

Another preferred embodiment provides a method of detecting a suspected chromosomal abnormality in subject-derived genomic or complementary nucleic acid. Generally, the method comprises the steps of preparing a spectrally-encoded, fluorescent microsphere having a first spectral address, identifying a target genomic nucleic acid probe sequence by ascertaining the nucleotide-by-nucleotide sequence of a target nucleic acid sequence wherein the abnormality is suspected to reside, synthesizing a low copy target probe according to the identified target genomic nucleic acid probe sequence, conjugating the target probe to a microsphere having a first spectral address, synthesizing a reference probe selected to hybridize to a reference nucleic acid sequence having a known copy number of said target nucleic acid sequence, conjugating the reference probe to a microsphere having a second spectral address, reacting the target probe with a chromosomal target sequence containing the abnormality thereby causing the target probe to hybridize to the target sequence, reacting the reference probe with a chromosomal reference sequence containing said chromosomal target sequence and thereby causing the reference probe to hybridize to the reference sequence, detecting the hybridized target probe to ascertain the existence of the chromosome abnormality, detecting the hybridized reference probe, and quantifying the detected hybridized target probe by comparing the response of the detected hybridized target probe with the response of the detected hybridized reference probe response. Preferably, said abnormality is selected from the group consisting of differences in copy numbers of sequences within said genomic or complementary nucleic acid, duplications, deletions, inversions, transpositions, translocations, and combinations thereof. In preferred forms, said method is effective at detecting said abnormalities with a genomic resolution of as little as 60 bp. The preferred method uses flow cytometry. Preferably, said low-copy probe has a copy number of 10, 9, 8, 7, 6, 5, 4, 3, 2, 1 or 0, depending upon the application or target nucleic acid sequence.

In another preferred embodiment, the present invention provides a method of direct genomic quantitation of chromosomal abnormalities via flow cytometric detection of labeled target nucleic acid sequences hybridized to microsphere-conjugated low copy genomic or complementary nucleic acid probes. Generally, the method comprises the steps of preparing a spectrally-encoded, fluorescent microsphere having a diameter between 50 nm and 5.8 μm, synthesizing a low copy target probe selected to hybridize to a particular nucleic acid sequence in the target wherein the abnormality is suspected to reside, conjugating the target probe to the microsphere, hybridizing the target probe to a target nucleic acid sequence, detecting the hybridized target probe via flow cytometry, and quantitating said abnormalities based on the results of said flow cytometry. Preferably, the nucleic acid sequence of a probe is as defined above. Such a method is particularly preferred for detecting and quantifying an abnormality selected from the group consisting of differences in copy numbers of sequences within said genomic or complementary nucleic acid, duplications, deletions, inversions, transpositions, translocations, and combinations thereof. As with the other preferred methods, this method can detect abnormalities with a genomic resolution of as little as 60 bp.

In another preferred embodiment, the present invention provides a method of detecting chromosomal abnormalities. Generally, the method comprises the steps of preparing a hybridization probe by coupling a spectrally-encoded, polystyrene microsphere to a synthetic DNA sequence, hybridizing said probe to genomic DNA, and detecting the product of said hybridization by flow cytometry. Detectable abnormalities and resolution are as described above. In preferred forms of this embodiment, an additional step of comparing the results from said flow cytometry with flow cytometry results from a hybridization product having a known copy number corresponding to said abnormality. Preferably, the probe is low copy or single copy. Advantageously, the DNA for this and the other preferred embodiments can be unamplified.

### DEFINITIONS

Unless defined otherwise, all technical and scientific terms used herein have the same meaning as is commonly understood by one of ordinary skill in the art to which this invention belongs. All patents, applications, published applications and other publications and sequences from GenBank and other databases referred to herein are incorporated by reference in their entirety. If a definition set forth in this section is contrary to or otherwise inconsistent with a definition set forth in applications, published applications and other publications and sequences from GenBank and other data bases that are herein incorporated by reference, the definition set forth in this section prevails over the definition that is incorporated herein by reference.

As used herein, “a” or “an” means “at least one” or “one or more.”

As used herein, “nucleic acid (s)” refers to deoxyribonucleic acid (DNA) and/or ribonucleic acid (RNA) in any form, including inter alia, single-stranded, duplex, triplex, linear and circular forms.

As used herein, the term “reference probe” means a probe specific for a locus in the genome, preferably from an autosomal sequence, that is severely damaging and preferably lethal in any other copy number but 2. The reference probe may be derived from any low or single copy chromosomal locus, so long as it has a normal chromosomal complement in the patient sample. In determination of genomic copy number for diagnosis of constitutional disease, reference probes will typically be of autosomal origin from one or more genes that are required to be expressed from two alleles during normal development. For determination of genomic copy number for diagnosis of neoplastic disease, reference probes are selected from chromosomal domains with a paucity of oncogenes and which have normal chromosomal complement.

As used herein, “label” refers to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The “label” may also be a compound that is a member of a binding pair, the other member of which bears a detectable physical property. Exemplary labels include mass groups, metals, fluorescent groups, luminescent groups, chemiluminescent groups, optical groups, charge groups, polar groups, colors, haptens, protein binding ligands, nucleotide sequences, radioactive groups, enzymes, particulate particles and a combination thereof.

As used herein, “sample” refers to anything that may contain an target nucleic acid to be analyzed. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid, or the like. Biological tissues are aggregates of cells, usually of a particular kind together with their intercellular substance that form one of the structural materials of a human, animal, plant, bacterial, fungal or viral structure, including connective, epithelium, skin, muscle and nerve tissues. Examples of biological tissues also include organs, tumors, lymph nodes, arteries and collections of individual cell(s), for example, isolated from plasma, blood or urine or by collagenase treatment of solid tissues.

As used herein, “amplification” refers to a method for exponentially duplicating a target analyte nucleic acid in a sample to improve assay sensitivity. As described herein, many different methods for amplifying nucleic acids are known in the art.

As used herein, “set” refers to a collection of microspheres harboring an identical spectral address conjugated either with a single lc probe or a collection of lc probes.

As used herein, “low copy” or “lc” refers to a sequence which will hybridize to ten or fewer sequence intervals in the target nucleic acid or locations in a genome. It is preferred that the copy number be 10 or fewer, more preferably 7 or fewer, still more preferably 5 or fewer, and most preferably 3 or fewer.

As used herein, “single copy” or “sc” refers to a nucleic acid sequence which will hybridize to three or less sequence intervals in the target nucleic acid or locations in a genome. Thus, the term will encompass sequences that are strictly unique (i.e., sequences complementary to one and only one sequence in the corresponding genome), as well as duplicons, and triplicons.

As used herein, “highly reiterated” means present in more than 10 copies.

### BRIEF DESCRIPTION OF THE DRAWING FIGURES

FIG. 1 is a schematic of a low copy probe-coupled microsphere hybridization assay;

FIG. 2 is a comparison of geometric mean ratios (x-axis) for pairs of low copy test probes (16-1b, 16-2b, PMP22, TEKT3) to reference probe (HOXB1c) compiled from 170 reactions;

FIG. 3 contains histograms of the fluorescence detection of multiplexed low copy probe-microspheres hybridized to samples A) intact for 16-2b (Sample 47, 16-2b:HOXB1 ratio=0.86, Table 2), B) duplicated for TEKT3 (CMT1A-1, TEKT3:HOXB1 ratio=1.40, Table 2) and C) deleted for 16-1b (Sample 33, 16-1b:HOXB1 ratio=0.49), and using HOXB1 as the internal genomic reference probe for each sample;

FIG. 4 is a map of PWS/AS Region on Chromosome 15q11-13; and

FIG. 5 is a summary of all QMH MFI ratios obtained in Example 2.

### DETAILED DESCRIPTION OF THE PREFERRED EMBODIMENTS

The following example sets forth preferred embodiments of the present invention. This example demonstrates detection of hemizygosity, trisomy, duplication, and insertion in the human genome and quantitation of nucleic acids in solution. However, this example is for illustrative purposes only and the disclosure herein should not be construed as a limitation upon the scope of the present invention.

### Example 1

### Materials and Methods

### Selection, Coupling and Hybridization of Microsphere-Conjugated Probes

Probe selection. Five lc probes were developed (Rogan et al, 2001; Knoll and Rogan, 2004) and used in this study to distinguish genomic copy number differences in the patient samples. They include: (i/ii) chromosome 9q34 probes (16-1 and 16-2) from within intron 1b of ABL1 and deleted in a subset of chronic myelogenous leukemia (CML) patients, (iii/iv) chromosome 17p12 probes recognizing TEKT3 and PMP22 which are within the CMT1A duplicated region (Inoue et al, 2001), and (v) a reference chromosome 7p15 probe recognizing HOXB1 (2 copies per diploid genome). The probe lengths, hybridization temperatures, and genome coordinates are listed in Table 1. Each probe had a copy number of one as determined for the entire length of the probe found in the genome; however, some homologous low copy sequences were found within small (<200 bases) segments of the probes.

Low copy (lc) probe synthesis. A single primer of each lc probe-specific pair (22-24 nucleotides in length) was synthesized with a 5′amino-modifier C-12 for coupling to microspheres (Integrated DNA Technologies, Coralville, Iowa). Polymerase chain reaction (PCR) using Pfx (Invitrogen, Carlsbad, Calif.) was performed with each primer pair (modified and unmodified) using control human genomic DNA (Promega, Madison, Wis.) as the template for lc probe synthesis. PCR products were separated by electrophoresis in low EEO agarose (Seakem, FMC Bioproducts, Rockland, Me.) and extracted by micro-spin column centrifugation (Qiagen, Valencia, Calif.). Products were quantitated using a spectrophotometer and subsequently conjugated to microspheres with an average of 120,000 DNA molecules coupled to each microsphere.

Coupling of probes to microspheres. Fluorescent microspheres, each with distinct spectral addresses (designated R1-R9; Molecular Probes, Eugene, Oreg.) and coated with approximately 200,000 carboxy-sites, were conjugated individually to different lc probes. Purified amino-modified lc probes were coupled to the carboxylated microspheres via a modified carbodiimide coupling procedure (Dunbar et al, 2003; Fulton et al, 1997). Each probe was initially heat denatured and then snap-cooled on ice. Approximately 3.125×10⁵ microspheres with identical spectral characteristics were pipetted into a 1.5 mL microcentrifuge tube (USA Scientific, Ocala, Fla.), centrifuged for 2 minutes at 10,000 g, and drained of supernatant. 150 μL of 0.1M MES buffer (2-(N-morpholino)ethanesulfonic acid) pH4.5 was added to each tube and the microspheres were vortexed briefly followed by centrifugation for 2 minutes at 10,000 g. The supernatant was removed and the microspheres were resuspended by vortexing in 80 μL of 0.1M MES. A single lc probe (0.5 mmol) was added to each tube and mixed by vortexing. A 1.25 μL volume of fresh 10 mg/ml solution of 1-ethyl-3-3-dimethylaminopropyl carbodiimidehydrochloride (EDC) was added and the reaction was vortexed briefly and incubated in the dark for 30 minutes with occasional mixing. Mixing and incubation of EDC was repeated twice, using 1.25 μL of freshly prepared EDC solution each time. The reaction was stopped by the addition of 500 μL 0.02% Tween20 followed by vortexing and centrifugation for 2 minutes at 10,000 g. Following removal of the supernatant, 250 μL of 0.1% SDS was added to each tube, vortexed, and then centrifuged at 10,000 g for 2 minutes. The supernatant was carefully removed, 25 μL of 0.1M MES pH4.5 was added, and the tube was vortexed and stored in the dark at 4° C. Coupled microsphere concentrations were quantitated by adding 1 μL of each microsphere to 100 uL of 1×PBS and analyzing on the FACSCalibur flow cytometer (Becton Dickinson, San Jose, Calif.) using conditions given below.

### Genomic DNA Template Preparation

Genomic DNA template was prepared from methanol-acetic acid fixed cell pellets derived either from patient samples remaining after clinical cytogenetic characterization or from cell lines obtained from the NIGMS-Coriell Cell Repository (Camden, N.J.). The fixed cells were washed twice with 1×PBS and their concentrations were determined with a hemocytometer. Genomic DNA template was extracted from ˜600 fixed cells per sample. This DNA was replicated in vitro using the GenomiPhi kit (Qiagen, Valencia, Calif.), and then nick-translated (1 μg) with biotin-16 dUTP for 60 minutes at 15° C. to obtain labeled products of 300 bp to 1 kb in length (Knoll and Lichter, 1994). 50 ng of nick-translated patient sample was analyzed in each hybridization assay.

The samples utilized to validate the method were known to exhibit differences in copy number for chromosome 9q34 and chromosome 17p12 based on routine cytogenetic analysis and/or fluorescence in situ hybridization (FISH). The cytogenetic findings for these test samples are listed in Table 2.

The samples included five from patients with CML, of which two had a concerted chromosome 9q34 ABL1 deletion (found in 10% of patients; Sinclair et al, 2000) in addition to the characteristic chromosome 9;22 translocation (designated Samples 33, 81) and three had only the chromosome 9;22 translocation with no deletion (designated Samples 38, 47, and 86); two from cell lines with complete trisomy 9 (GM09286 and GM10186); one from a cell line with a chromosome 9q34 duplication (GM06074); and 5 from patients with Charcot-Marie-Tooth Disease type 1A and a chromosome 17p12 duplication (designated CMT1A-1 through 4, and GM12214). Institutional Review Board approval was obtained for use of the residual fixed cells.

Hybridization reactions. For hybridization, 50 ng of each sample was diluted in 40 μL TMAC hybridization buffer (3-mol/L tetramethylammonium chloride, 50 mmol/L Tris-HCl, pH8.0, 1 g/L sarkosyl) containing 5,000 lc probe-coupled microspheres. The hybridization reactions were heat denatured at 95° C. for 3 minutes and then hybridized overnight at 45° C. to 51° C. depending upon the probe nucleotide composition and length (Lewin, 1980) (Table 1). The hybridized microspheres were then washed with 250 uL of 1.5×TMAC (Dunbar et al, 2003) followed by centrifugation at 10,000 g for 2 minutes. The supernatant was removed and 12 μL of a 1:50 dilution of a reporter molecule, streptavidin-phycoerythrin (SPE; Molecular Probes), in 1.5×TMAC was added to detect genomic targets containing biotin. The reactions were incubated at their hybridization temperature for 12 minutes. Following labeling, 250 μL of 1.5×TMAC was added to each reaction, mixed, and centrifuged at 10,000 g for 2 minutes. The supernatant was removed and the hybridized microspheres were resuspended in 70 μL of 1.5×TMAC.

Flow cytometry detection of hybridized microsphere-coupled low copy probes. Hybridization reactions were diluted in 300 μL 1×PBS prior to analysis on a FACSCalibur flow cytometer. Approximately 5,000 microspheres of each set were analyzed per reaction. The signal of each target, hybridized to its complementary probe coupled to microspheres, was determined from the fluorescence intensity of SPE. Compatible microsphere spectral addresses selected to minimize overlap with the emission wavelengths of phycoerythrin (PE) were confirmed by comparing results obtained with otherwise identical unconjugated and hybridized microspheres. For each reaction, a reaction tube with all the components except target DNA was used as a negative control to determine background fluorescence in the FL2 (PE) detection channel. Fluorescent bead standards (LinearFlow Flow Cytometry Intensity Calibration Kit; Molecular Probes) were used for intensity calibration of different fluorochrome detection channels of the flow cytometer. The instrument was also calibrated with fluorescent reference standards (Quantum R-PE MESF Medium Level Kit; Bangs Laboratories, Fishers, Ind.), based on surface-labeled beads calibrated in molecules of equivalent soluble fluorochrome (MESF) units.

Optimal photomultiplier tube voltage settings were determined by selecting photomultiplier voltage tube settings that minimized differences between fluorescence intensities of two different probes hybridized to a single patient DNA sample with a normal genotype. These settings were determined from instrument-derived fluorescence measurements (CellQuest; Becton Dickinson) of arithmetic mean, geometric mean, median and peak channel. Typical photomultiplier tube voltage settings for the FACSCalibur instrument were FSC (forward scatter)=E00 (no signal amplification), SSC (side scatter)=344 V, FL1=727 V, FL2=640 V, FL3=300 V, and FL4=500 V. Thresholds for FSC, FL1, FL2, and FL3 were set at the default of 52 V. The FSC threshold was selected as the primary parameter and had a value of 52 V and the secondary parameter was set at SSC with a value of 125 V. The flow rate was set on low and the sheath fluid used was FACsFlow (Becton Dickinson). The system was flushed between runs with 2-5 mL of distilled water to remove any residual microspheres. CellQuest was used for data collection and analysis. Analysis of data was also performed using WinMDI2.8 flow cytometry package (WinMDI; J. Trotter, Salk Institute, La Jolla, Calif.).

Most of the probes tested did not require significant optimization for composition because probe quality depended mainly on the low copy nature of the sequence used to design it. The fluorescence intensity ratios fell outside of established confidence intervals or known genotypes for sequences that were paralogous to other genomic loci (data not shown). This was circumvented by using lc probes that were strictly not homologous to other genomic segments. The optimal voltage settings resulted in tightly clustered microspheres with different spectral addresses, as visualized with CellQuest using the sidescatter (SSC) plot. Non-optimal voltage parameters resulted in broad FL2 peaks or non-linear data (Bagwell et al, 1989; Brown et al, 1994).

We first showed that all probes could detect the corresponding PCR product in a hybridization reaction, thereby establishing that probe conjugation and hybridization to microspheres were efficient. Purified ABL1 (16-1, 16-2), TEKT3, PMP22, and HOXB1 products were amplified from normal genomic DNA (Promega) using a single 5′ biotinylated primer. The biotinylated PCR products were hybridized to complementary microsphere-conjugated probes and detected with SPE. Hybridized microsphere count and mean fluorescence intensities were measured using CellQuest software (Becton Dickinson). The density of hybridized lc products for a set of microspheres was estimated by comparing the quantity of single beads (from the gated side scatter bead count) to the number of hybridized beads (gated bead signal count and mean fluorescence intensity level), which determined the conjugation efficiency of the carbodiimide coupling procedure. The specificity of the microsphere-conjugated lc probe to its target sequence was determined by comparing mean fluorescence intensity of repeated flow cytometry runs of independent hybridization assays with homologous versus heterologous probes. Hybridization of each probe was optimized across a range of annealing temperatures (45° C. to 60° C.).

### Verification of Probe Specificity and Sensitivity

Genomic reconstruction experiments. To test the specificity of lc probes in a complex genomic environment, lc probe-coupled microspheres were hybridized to their corresponding purified PCR products in the presence of excess sheared calf thymus DNA (Amersham Biosciences, Piscataway, N.J.). Independent hybridization reactions were performed for 16-1a, 16-2a and HOXB1a and were carried out with 10,000 microspheres for each probe, 5 ng of the corresponding PCR product, and 50 ng of nick-translated calf thymus genomic DNA. To estimate the lowest detectable amount of hybridized product, a dilution series of PCR products (5-150 genomic equivalents per hybridization reaction) were added to 10 ng of sheared calf thymus DNA with a molar ratio of PCR product:calf thymus DNA ranging from ˜1:300 to 1:10.

Multiplex hybridizations with lc probe-conjugated microspheres for 16-1a, 16-2a, and HOXB1a and 5 ng each of their corresponding PCR products were also performed in the presence or absence of 10 ng of sheared calf thymus genomic DNA. Additionally, microsphere swap experiments were performed in which lc probe 16-1a was conjugated to two microsphere sets with different spectral addresses (R2 and R9), hybridized to corresponding PCR products in a multiplex reaction, and mean fluorescence intensity levels were compared for each microsphere set.

Statistical analysis was performed using MATLAB (MATHWORKS, Natick, Mass.). Mean fluorescence ratios between test and reference probes are reported using arithmetic means in text (Coder et al, 1994). Residuals were calculated by fitting mean fluorescence ratios and peak channel values to expected values based on known genotypes.

### Results

### Specificity of the Hybridization Assay

PCR products homologous to lc probes coupled to microspheres were used as target DNA in hybridization reactions to examine the labeling efficiency of the carbodiimide coupling procedure. Based on the SPE mean signal (in the FL2 channel) above background fluorescence (>10¹) and the number of microspheres hybridized to PCR products present in the reaction, 98% (+/−0.4%) of the microspheres had hybridized to target PCR product (data not shown).

Genomic reconstruction experiments confirmed that lc probes 16-1a, 16-2a, and TEKT3 are not homologous (>75%) to any sequence in the calf (Bos taurus) genome. Based on sequence analysis, the Bos taurus genome should not contain sequences closely related to probes 16-1, 16-2, TEKT3 and PMP22. Mean fluorescence intensities below background levels were consistently observed for these SPE signals. By contrast, HOXB1a showed a mean fluorescence intensity of 12.81 in one hybridization assay, which was expected since this probe is 90% homologous to HOXB1 sequences in the Bos taurus genome.

Conjugated lc probes 16-1a and 16-2a detected homologous sequences seeded into a heterologous genomic background. The sensitivity of detection was linearly related to the amount of probe present, based on increases in SPE mean fluorescence intensity with product concentration (data not shown). Microsphere swap experiments showed that these results were independent of the spectral address of the microsphere. The mean hybridization signal intensities for different microsphere intensities conjugated to the same probe gave very similar values in signal intensity. For example, for Sample 47, multiplex hybridizations of probe 16-1a conjugated to microsphere R2 yielded a mean fluorescence intensity of 32.8, and microsphere R9 gave a mean fluorescence signal of 32.02, a difference of ˜2%. With Sample 33, the mean fluorescence of this same probe varied by ˜13% (41.88 vs. 36.16) for microspheres with different spectral addresses.

### Optimization of Probe Length

Probes of varying lengths were conjugated to microspheres to examine the effect of probe length on the hybridization signal. Lc probes 16-2a (1381 bases) and 16-2b (101 bases), which are subsets of probe 16-2, were coupled to the surface of spectrally distinct microspheres and multiplexed in a single hybridization reaction to Sample 47 (which has a normal genotype at the 16-2 locus). Surprisingly, the mean fluorescence signal ratio for the shorter 16-2b probe to HOXB1c was approximately double the corresponding 16-2a ratio. A multiplex experiment with DNA from a chromosome 9 deletion patient (Sample 33) showed 16-2a and 16-2b probes to have lower signals—52% and 66%, respectively than the HOXB1c reference. Since the deletion patient has half the number of copies of 16-2 compared to HOXB1c, the signal obtained with the shorter 16-2b probe appears to more precisely reflect the actual copy number in these individuals. Similar results were obtained with subsets of probe 16-1 (16-1a [2304 bp] and 16-1b [100 bp]) conjugated to microspheres when independently hybridized to the corresponding PCR product and control genomic DNA. Both probes successfully hybridized to both PCR product and control genomic DNA, however the SPE fluorescence distributions for the 16-1a reactions were much broader than they were for 16-1b. We found that microspheres conjugated to the shorter probes (˜100 bases) produced well-defined mean fluorescence distributions and consistently higher PE values. These conjugates could also be used in hybridization reactions for more than two months after conjugation when stored at 4° C. in the dark, unlike longer conjugated probes which showed degraded hybridization efficiency within 2 weeks of preparation.

Shorter oligonucleotide probes (HOXB1e and 16-1c) proved to be adequate for characterizing genotypes of known samples. Such probes produced test to reference probe MFI ratios that could distinguish copy number. For instance, DNA from the trisomy 9 cell line, GM09286, was hybridized with test probe 16-1c (62 bases) and reference probe HOXB1e (62 bases), yielding a MFI ratio of 1.28. This MFI ratio is consistent with the presence of 3 copies of the 16-1c sequence.

Detection of Reduced Genomic Copy Number in Samples with a Deletion of 9q34

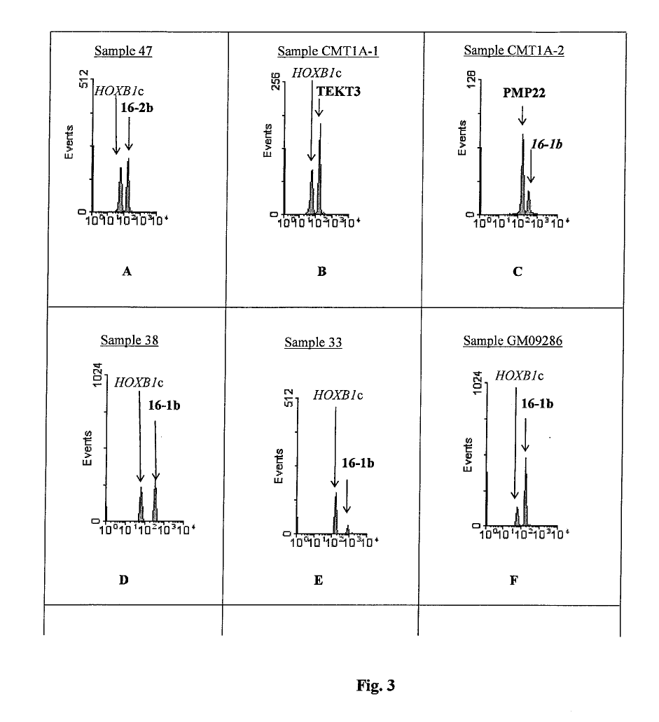

The genotypes of chromosome 9q34 deletion and normal control samples were distinguishable based on comparisons of fluorescence intensities. In a blind study, microspheres conjugated to 16-2b and HOXB1c were hybridized in separate reactions to Sample 33 and Sample 47 genomic DNA (FIG. 3). In the histograms of FIG. 3, the fluorescence intensity signal (FL2 phycoerythrin, x axis) versus the microsphere count (y-axis) is plotted for the test probe and the reference probe for each sample. The ratio of the mean fluorescence of the 16-2b probe to the HOXB1c reference probe (Table 2) indicates a lower copy number for 16-2b in Sample 33 (ratio=0.67), consistent with a deletion of one copy of this locus, which was previously demonstrated by scFISH with probe 16-2a. Similar signal intensities were found for both probes in Sample 47 (ratio=0.96), consistent with a normal genotype. The mean fluorescence ratios of probes 16-1b to HOXB1c in Samples 33 and 81 were also consistent with FISH results demonstrating a deletion of one copy of 16-1a in both patients.

Samples 33 and 38 were then hybridized with probes 16-1b, 16-2b, and HOXB1c in separate multiplexed reactions. Relative to the HOXB1c probe, Sample 33 showed mean fluorescence of 61% for 16-1b and 51% for 16-2b. Sample 38 hybridizations showed similar HOXB1c and 16-1b (87%) and 16-2b (89%) mean fluorescence levels. These results are consistent with the FISH data that demonstrate that Sample 33 has a hemizygous deletion for ABL1 and Sample 38 does not.

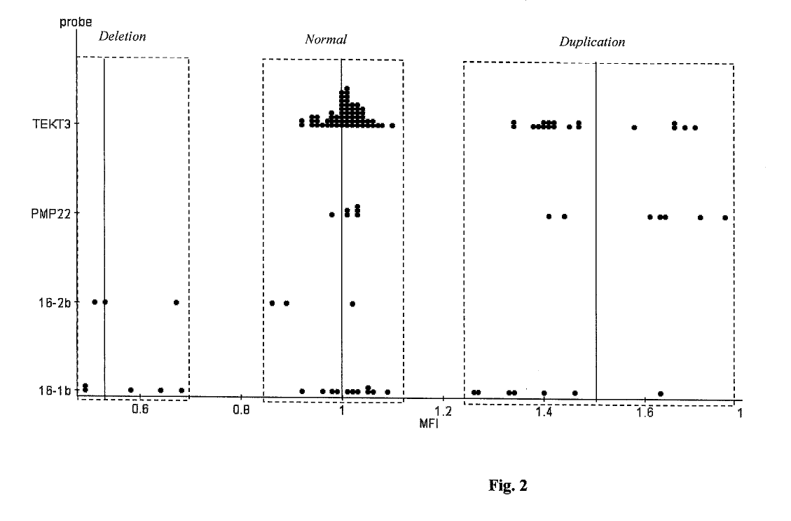

The ratio of the signal intensity of 16-1 to HOXB1 and 16-2 to HOXB1 for samples with a 5′ABL1 deletion and for those without a deletion are graphically depicted in FIG. 2. In FIG. 2, samples are grouped by test probe (y-axis). A box (dashed lines) is drawn around each distinct genotypic group (left=chromosomal deletion, middle=normal, right=chromosomal duplication). Vertical lines indicate the theoretical MFI ratio for each genotype (0.5, 1.0, and 1.5 for deletion, normal and duplicated samples, respectively). As indicated, the MFI ratios for all samples within each genotype do not overlap with other genotypes. In general, all of the samples tested with a disomic complement have ratios for 16-1 or 16-2 derived probes relative to HOXB1 mean fluorescence intensities close to 1, and those samples with a 5′ABL1 deletion have ratios close to 0.5 (Table 5). The geometric mean and median provide the most accurate measures of fluorescence intensity for signal in our hybridization assay, since these metrics have the smallest residual variance (0.10) in regression against the expected ratios for all of the genotypes studied.

### Detection of Increased Copy Number of Chromosome 9q34 in Trisomic 9 Cell Lines

To determine if triallelic copy number could be distinguished from biallelic copy number, probe 16-1b was hybridized to DNA from cell lines trisomic for 9q34 and compared with Sample 47, which is disomic for this region (Table 2). Both HOXB1c and 16-1b were multiplexed in a single hybridization reaction. Cell lines GM09286 (ratio=1.34), GM10186 (ratio=1.40) and GM06074 (ratio=1.26) each exhibited significantly higher mean fluorescence for 16-1b compared to HOXB1c, whereas both probes exhibited similar mean fluorescence signals for Sample 47 (ratio=0.96). We suggest that a deviation from the expected ratio of 1.5 may be due to either the stability, i.e. low guanine-cytosine (GC) content, of lc probe 16-1b, or to the secondary structure of the genomic region tested, resulting in fewer microsphere-bound targets. The assay confirms previous cytogenetic and FISH results showing the additional copy of the chromosome 9q34 sequence in all three cell lines and the normal copy number of the sequence in Sample 47 (Table 5).

### Detection of Increased Copy Number of Chromosome 17p12 Region in CMT1A Patients

Genomic DNA from five CMT1A patients with FISH-confirmed chromosome 17p12 duplications and Sample 86 with a normal karyotype in this chromosomal region were hybridized with either TEKT3 or PMP22 and HOXB1c lc probes conjugated to microspheres. All CMT1A patient samples displayed similar elevations in mean fluorescence intensity signals (ranging from 28-40% greater than HOXB1c) for hybridization to TEKT3, reflecting the presence of three copies of this locus (FIG. 3). Sample 86 exhibited only a 2% difference in mean fluorescence signal intensities for the two probes (Table 2). Similar results were obtained for a multiplex analysis of the same CMT1A samples tested with PMP22 and HOXB1 (ranging from 28.5 to 54% above HOXB1c; see Table 2). Independent multiplex hybridizations with microsphere-coupled TEKT3 and HOXB1 hybridized to CMT1A-1 and CMT1A-2 demonstrated that these results were reproducible, yielding mean fluorescence intensity ratios that varied by less than 2% for CMT1A-2 and by 16% for CMT1A-1 (Table 2). These ratios indicate a clustering around the expected value of 1.5 (1.26-1.85) for samples containing 17p12 duplications (FIG. 2) (Table 5).

The geometric mean or median fluorescence ratio was found to be the best measure for the hybridization assay because it provided the smallest confidence intervals and residuals for all of the genotypes with no overlap between genotypes (FIG. 2). Multiple linear regression of data for each ratio category against expected ratios over all genotypes gave correlation coefficients of 0.86 for the arithmetic mean, 0.89 for geometric mean and median, and 0.42 for the peak channel ratio. The 95% confidence intervals for the geometric mean fluorescence ratio of test to reference sample intensities for specimens with normal diploid genotype was 0.97 to 1.03, for hemizygous deletions was 0.5 to 0.64, and for duplications was from 1.44 to 1.56. The arithmetic mean produced slightly larger confidence intervals (residual variance=0.13), and the peak channel fluorescence was found to be a suboptimal measure of copy number, because several hybridization reactions produced much larger residuals for these genotypes.

In a blinded study, triplicate reactions of 22 coded genomic samples (consisting of 20 normal and 2 CMT1A patient samples with previously confirmed duplications) were hybridized to TEKT3 and HOXB1c probes. Reproducibility for triplicate runs of each of these samples was excellent, with variances in the MFI ratios ranging from 6.5×10⁻⁶ to 1×10⁻³. Patient samples with CMT1A duplications were readily distinguishable from normal controls, based on the respective TEKT3:HOXB1c hybridization MFI ratios for these groups (summarized in Table 5). These are consistent with our previous results showing no overlap between these genotypes in our non-blinded analysis (FIG. 2).

### Distinguishing Copy Number Genotypes

The MFI ratio is a robust metric for determining genomic copy number, regardless of which loci are analyzed. The average test to reference probe MFI ratio was 1.0±0.06 (n=21) in all normal individuals in the present study (Table 2). The ratio was 0.57±0.08 (n=8) for all samples with hemizygous deletions, and was 1.50±0.15 (n=26), for triallelic samples. While several probes showed a modest degree of skewing of MFI ratios, abnormalities were clearly distinguishable from normal genotypes for all of the lc probes at the 95% confidence level, and for the normal versus deleted genotypes, at the 98% confidence level.

### Discussion

Suspensions of spectrally-encoded polystyrene microspheres, coupled to synthetic DNA sequences, can be hybridized to genomic DNA and detected by conventional flow cytometry instruments. This approach has facilitated high throughput genotyping of single nucleotide polymorphisms (Vignali, 2000). However, prior art methodologies required that the target DNA be amplified prior to hybridizing the target DNA to a 10-50 nucleotide oligonucleotide probe coupled to microspheres (Hadd et al, 2004; Rockenbauer et al, 2005). Oligonucleotide probes cannot accurately quantify genomic copy number directly from patient DNA samples (Earley et al, 2002; Sekar et al, 2005; Vignali, 2000) because of inadequate sensitivity and specificity. Copy number estimation of target sequences is also complicated by the fact that the requisite amplification step can be difficult to control because it is inherently logarithmic. Genomic copy number can be unequivocally determined when directly-labeled genomic DNA is hybridized without prior amplification (Southern, 1975; White et al, 2004).

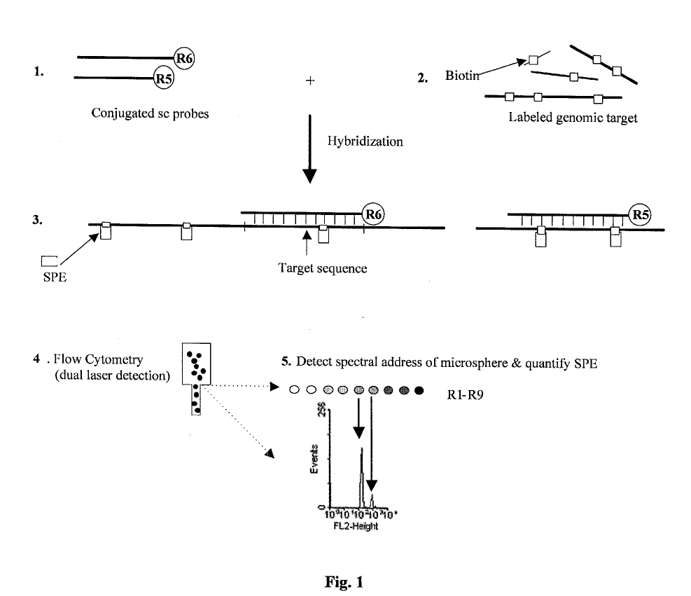

We examined the possibility that direct hybridization of patient-derived DNA to longer probes coupled to microspheres could quantitatively measure copy number (FIG. 1). In step 1) of this figure, each lc probe is synthesized by PCR using an amino-modified forward primer and conjugated to spectrally distinct carboxylated microspheres. In step 2), sample DNA is extracted from fixed cytogenetic cell pellets and nick-translated to incorporate biotin dUTP. Step 3) illustrates that lc probe-coupled microspheres are hybridized to the prepared genomic DNA and stained with streptavidin-phycoerythrin (SPE) and washed to remove residual SPE. In step 4), samples are run on the flow cytometer (dual laser detection) for the detection of distinct spectral addresses of microspheres and quantification of target bound by each microsphere-coupled probe in step 5). Previously, we validated computationally-derived low copy (lc) probes to detect a wide variety of chromosomal abnormalities by fluorescence in situ hybridization (FISH) for many different chromosomal regions (Knoll and Rogan, 2003; Rogan et al, 2001). In this study, we developed microsphere suspension hybridization with lc probes from three different chromosomal regions associated with aneusomic conditions and demonstrated the use of this method to detect copy number gains or losses in archival, methanol-acetic acid fixed cytogenetic specimens.

With the present invention, a microsphere suspension hybridization assay to detect genomic copy number differences was developed which utilizes low copy (lc) genomic probes. Prior amplification of locus-specific target DNA was not required since lc probes are designed to hybridize to a unique locus in the haploid genome sequence with high specificity. Loss or gain of low copy sequences can be directly detected in patient genomic DNA. This assay can follow routine cytogenetic analyses without requiring large patient sample quantities, additional blood draws, locus-specific genomic amplification or time-consuming genomic DNA purification methods. These studies were performed using fixed cell preparations remaining after cytogenetic analyses.

Hybridization experiments demonstrated adequate sensitivity to discern the presence of one versus two copies as well as two versus three copies of a genomic sequence. Use of multiple independent lc probes conjugated to microspheres with distinct spectral signatures can independently measure copy number changes in the same hybridization assay. Chromosome deletions were confirmed in the ABL1 gene in two CML patients, trisomy of chromosome 9q34 was confirmed in three cultured cell lines, and duplication of chromosome 17p12 was confirmed in cells of five CMT1A patients, including one cell line. The same probe used to detect a hemizyogous chromosome 9q34 deletion also detected three alleles in cell lines with trisomy at this locus. Ratios of probe mean or median fluorescence intensities were consistent for patients with the same genotype and were clustered around expected values, regardless of the chromosomal origin of the test probe.

Several parameters were optimized during assay development: (i) the lc probes conjugated to microspheres showed specificity for homologous sequences when examined in a heterologous complex genomic environment, i.e. as little as 5 ng of target sequence present in 1 ug of heterologous genomic sequence was successfully detected; (ii) lc probes conjugated to microsphere sets with different spectral addresses showed negligible differences in mean fluorescence intensities, regardless of which microsphere was conjugated to a probe; (iii) the length of the lc probe attached to the microsphere surface affected the efficiency of hybridization, with shorter probes (˜100 bp) exhibiting greater mean fluorescence intensities compared to longer probes (1 to 2 kb). The shorter probes were more stable (longer shelf life) resulting for less lot-to-lot variation in labeled microsphere stocks as well as (iv) this, in turn, reduced the effort required to conjugate, quantify and qualify DNA-bound microspheres.

### Example 2

This example describes a clinical application of quantitative microsphere hybridization to examine the Prader-Willi (PWS) and Angelman syndrome (AS) region on chromosome 15q11-13. 17 low copy and single copy test probes (80 nucleotides each) spanning ˜3.2 Mb of the PWS/AS region and a disomic reference probe (HOXB1, chromosome 17q21), were each conjugated to one of ten spectrally distinct polystyrene microsphere levels. All probes were hybridized to biotin-labeled genomic patient DNA in multiplex QMH reactions, and hybridization was detected using phycoerythrin-labeled streptavidin and analyzed by dual-laser flow cytometry. Copy number differences were distinguished by comparing mean fluorescence intensities (MFI) of the test probes to the reference probe. The MFI ratios for deleted loci were 0.58±0.072 (n=80) as compared to the MFI ratios for normal loci, 0.98±0.075 (n=186).

Genomic rearrangements of chromosome 15q11-13 cause diverse phenotypes including autism, Prader-Willi syndrome (PWS), and Angelman syndrome (AS). This chromosomal region is subject to genomic imprinting and characterized by complex combinations of low copy repeat elements (refs). PWS and AS typically result from an ˜4 Mb deletion in the paternal or maternal chromosome 15q11-13 region (Nichols 1998), respectively, with clustered breakpoints (BP) at either of two proximal sites (BP1 and BP2) and one distal site (BP3) which enables the classification of deletion patients (class I and II) (Knoll et al. 1990). Class I deletion patients are deleted for the region spanning BP1 to BP3 and class II deletion patients are deleted for the region spanning BP2 and BP3 (Knoll et al. 1990). Additionally, mutations within the bipartite imprinting center (IC) in this region can cause either disorder. One region of this IC is required for establishing and maintaining the paternal or maternal imprint and studies have ascertained the shortest region of overlap (SRO) which defines the PWS-IC (PWS-SRO) and AS-IC (AS-SRO), (Mapendano et al. 2006). Further, both PWS and AS can arise from chromosome 15 uniparental disomy (UPD) which is maternal in origin in PWS patients (maternal disomy) or paternal in origin in AS patients (paternal disomy).

### Materials and Methods

Probe selection, synthesis and microsphere conjugation. A series of 17 different test lc probes specific to chromosome 15q11-13 were designed, as well as a disomic reference probe from HOXB1 on chromosome 17q21. Each of the probes was 80 bases in length as shown in Table 3. There was only one genomic match for the full 80 bases of each probe; however, small segments of probe may match other genomic regions.

Probes were selected based on their low copy sequence composition, GC content (48-55%), the lack of potential stable secondary sequence conformations as predicted by MFold software (available through The Bioinformatics Center at Rensselaer and Wadsworth, Rensselaer Polytechnic Institute, Troy, N.Y.), and their length (80 nucleotides). Each probe was synthesized using a 5′-C6-amino-labeled forward primer (Integrated DNA Technologies, Coralville, Iowa) for coupling to carboxylated microspheres, and an unmodified reverse primer by PCR (Promega RedTaq Ready Mix, Madison, Wis.) using a normal human genomic control DNA (Promega) as template. PCR product was separated by gel electrophoresis (Seakem; FMC Bioproducts, Rockland, Me.) and extracted by microspin column centrifugation (QiaQuik; Qiagen, Valencia, Calif.). Probes were conjugated to one of ten spectrally distinct microsphere levels (CytoPlex; Duke Scientific, Palo Alto, Calif.) via a carbodiimide coupling reaction (Dunbar et al. 2003; Fulton et al. 1997).

Each probe was initially heat denatured and then snap-cooled on ice. Approximately 3.125×105 microspheres with identical spectral characteristics were pipetted into a 1.5 mL microcentrifuge tube (USA Scientific, Ocala, Fla.), centrifuged for 2 minutes at 10,000 g, and drained of supernatant. 150 μL of 0.1M MES buffer (2-(N-morpholino)ethanesulfonic acid) pH4.5 was added to each tube and the microspheres were vortexed briefly followed by centrifugation for 2 minutes at 10,000 g. Supernatant was removed and the microspheres were resuspended by vortexing in 80 μL of 0.1M MES. A single lc probe (0.5 mmol) was added to each tube and mixed by vortexing. A 1.25 μL volume of fresh 10 mg/ml solution of 1-ethyl-3-3-dimethylaminopropyl carbodiimidehydrochloride (EDC) was added and the reaction was vortexed briefly and incubated in the dark for 30 minutes with occasional mixing. Mixing and incubation of EDC was repeated twice, using 1.25 μL of freshly prepared EDC solution each time. The reaction was stopped by addition of 500 μL 0.02% Tween20 followed by vortexing and centrifugation for 2 minutes at 10,000 g. Following removal of the supernatant, 250 μL of 0.1% SDS was added to each tube, vortexed and then centrifuged at 10,000 g for 2 minutes. The supernatant was carefully removed, 25 μL of 0.1M MES pH4.5 was added and the tube was vortexed and stored in the dark at 4° C. Coupled microsphere concentrations were quantitated by adding 1 μL of each microsphere to 100 uL of 1×PBS and analyzing on the FACSCalibur flow cytometer (Becton Dickinson, San Jose, Calif.) using conditions given below.

Quality control procedures for each microsphere-conjugated probe were performed to ensure proper attachment of probes to microspheres. It was first determined that all probes could detect the corresponding PCR product in a hybridization reaction, establishing that probe conjugation and hybridization to microspheres were efficient. Purified PCR products specific to each of the QMH probe utilized in this study (Table 3) were amplified from normal genomic DNA (Promega) using a single 5′ biotinylated primer. The biotinylated PCR products were hybridized to complementary microsphere-conjugated probes and detected with SPE. Hybridized microsphere count and mean fluorescence intensities were measured using CellQuest software (Becton Dickinson). The density of hybridized lc products for a set of microspheres was estimated by comparing the quantity of single beads (from the gated side scatter bead count) to the number of hybridized beads (gated bead signal count and mean fluorescence intensity level), which determined the conjugation efficiency of the carbodiimide coupling procedure. The specificity of the microsphere-conjugated lc probe to its target sequence was determined by comparing mean fluorescence intensity of repeated flow cytometry runs of independent hybridization assays with homologous versus heterologous probes. Hybridization of each probe was optimized across a range of annealing temperatures (45° C. to 60° C.).

Additionally, microsphere-swap experiments were performed to verify that the MFI value was independent of the level of microsphere used for conjugation, the details for which were stated previously (Newkirk et al. 2006). Such experiments were performed using lc probe D15S63 conjugated to two microsphere sets with different spectral addresses (L2 and L9). Both microsphere sets were hybridized to corresponding PCR product in a multiplex reaction, and mean fluorescence intensity levels were compared for each microsphere set. Both reactions showed similar MFI values for the same probe. This experiment was repeated for lc probe IC (Table 3) with similar results.

Patient samples and genomic target preparation. Genomic DNA template was prepared using archived patient DNA samples (n=21) or using methanol-acetic acid fixed cell pellets derived from cytogenetic preparations of bone marrow samples (n=10). Fixed cell pellets from patients had been stored from two years to eight years in methanol and acetic acid. Genotypes for some samples were known from prior cytogenetic, microsatellite, and/or Southern hybridization studies, while some were unknown (Table 4-7). The fixed cells were washed twice with 1×PBS and their concentrations were determined with a hemocytometer. Genomic DNA template was extracted from ˜600 fixed cells per sample.

For archived DNA samples, no treatment prior to amplification was necessary. Whole genomic DNA was replicated in vitro using a modified protocol of the GenomiPhi kit (GE Healthcare, Piscataway, N.J.) to allow for the direct incorporation of biotin into template DNA. First, 1 uL of each DNA sample was mixed with 9 uL of Sample Buffer (GE Healthcare) and heat denatured for 3 minutes at 95° C. Samples were snap-cooled on ice for 2 minutes followed by addition of 4 uL of 50 nmol Biotin-16-dUTP (Roche, Indianapolis, Ind.), 9 uL of Reaction Buffer (GE Healthcare), and 1 uL of Enzyme Mix (GE Healthcare). Reactions were incubated at 30° C. for 24-36 hours. Successful replication of patient DNA was verified by PCR amplification of the HOXB1 and PWS-SRO probes (Table 3). Any sample not yielding PCR product in this initial quality control step was not used for QMH. It was observed that samples that did not successfully amplify PCR product did not produce accurate QMH results (data not shown). The age of the fixed cell pellet is a likely contributing factor for the lack of PCR amplification following the modified GenomiPhi procedure. Analysis of these samples by PCR and gel electrophoresis indicated the extracted DNA was highly degraded and sheared, which would in turn cause an intact probe to appear deleted in QMH. It was observed that genomic DNA which passed the PCR quality control was obtained from more recently stored fixed cell pellets (˜2-5 years) versus older cell pellets (>6 years). DNA samples which passed the quality control PCR assay were sheared to ˜300-800 bp by sonication (B-300, Bransonic, Danbury, Conn.), which was monitored by gel electrophoresis (Seakem). Twenty five ng of each labeled and fragmented genomic DNA sample was used per QMH reaction.

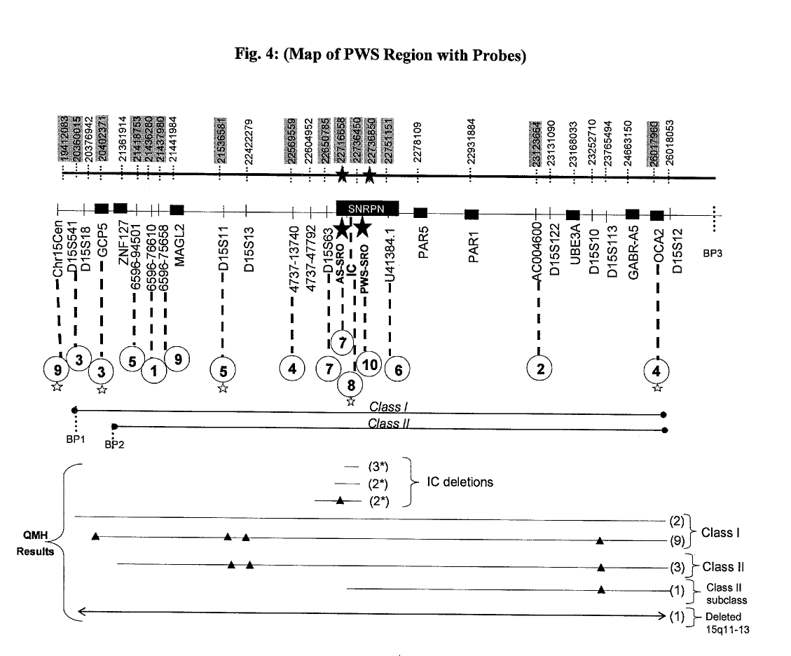

Hybridization reactions and flow cytometry. Prepared genomic DNA was diluted in 40 uL of 1.5×TMAC hybridization buffer (3-mol/l tetramethylammonium chloride, 50 mmol/l Tris-HCl, pH8.0, 1 g/l Sarkosyl) containing ˜7,500 of each probe-coupled microsphere. Multiplex analysis of two to ten different levels of microspheres conjugated to probes for QMH was possible in a single reaction using the CytoPlex microspheres (Duke Scientific; Palo Alto, Calif.). Probes included in hybridization reactions were selected based on available genotypic evidence in each previously characterized patient or family, and unknown samples were hybridized with chr15Cen, GCP5, D15S11, D15S63, PWS-SRO, and OCA2 initially in effort to delineate class I, class II, and IC deletions (Table 3). QMH probes used in subsequent hybridization reactions were selected adjacent to and within any deleted regions detected in the first QMH reaction in order to precisely characterize the deletion interval (FIG. 4). Reactions were heat denatured at 95° C. for 5 minutes and allowed to hybridize overnight at 50° C. Reactions were centrifuged for 2 minutes at 10,000 g, the supernatant was removed, and 12 μL of a 1:50 dilution of a reporter molecule, streptavidin-phycoerythrin (SPE; Molecular Probes, Eugene, Oreg.), in 1.5×TMAC was added to detect genomic targets containing biotin. The reactions were incubated at their hybridization temperature for 12 minutes. Following labeling, 250 μL of 1.5×TMAC was added to each reaction, mixed, and centrifuged at 10,000 g for 2 minutes. The supernatant was removed and the hybridized microspheres were resuspended in 70 μL 1.5×TMAC.

Samples were evaluated by flow cytometry analysis using a FACSCalibur (Becton Dickenson, San Jose, Calif.) and approximately 20,000 events were analyzed per reaction. Typical PMT voltage settings for the FACSCalibur instrument were FSC (forward scatter)=E00 (no signal amplification) in Log Mode, SSC (side scatter)=344V in Linear Mode, FL1=628V in Logarithmic Mode, FL2=620V in Logarithmic Mode, FL3=710V in Logarithmic Mode, and FL4=500V in Logarithmic Mode. Thresholds for FL1, FL2, and FL3 were set at a default of 52V. The FSC threshold was the primary parameter with a value of 50V and the secondary parameter was SSC and set at 130V. The flow rate was set on low. Color compensation was set as follows: FL1=1.9% FL2, FL2=21% FL1, FL2=0% FL3, FL3=10% FL2, FL3=4.7% FL4, and FL4=3.6% FL3. Color compensation values minimize the spectral overlap of MFI values measured in different channels of the flow cytometer. These settings were determined as optimal based on distinct clusters for each of the 10 different levels of unlabeled microspheres in the SSC vs. bead signal plot. Fluorescent bead standards (LinearFlow Flow Cytometry Intensity Calibration Kit; Molecular Probes) were used for intensity calibration of different fluorochrome detection channels of the flow cytometer. The instrument was also calibrated with fluorescent reference standards (Quantum R-PE MESF Medium Level Kit; Bangs Laboratories, Fishers, Ind.), based on surface-labeled beads calibrated in molecules of equivalent soluble fluorochrome (MESF) units. After analysis of each sample by flow cytometry, the ratios of geometric mean fluorescence intensities (MFI) of test probe to reference probe were calculated and statistics (eg. mean, 95% confidence intervals, and standard deviation) were generated using MS Excel as shown in Tables 4-1 through 4-9 (referred to collectively as Table 4). Tables 4-1 through 4-9 each include probe identifiers in the leftmost column followed by sample results in paired columns including mean fluorescence intensities and mean fluorescence intensity ratios. Data showing deletions is bounded by a darkened boarder. Geometric MFI values have been previously shown to be the most accurate for assessing data from QMH assays since data is collected in logarithmic mode on the flow cytometer (Kirkwood 1979; Coder et al. 1994; Newkirk et al., 2006).

### Results

### Detection of Large Deletions in PWS/AS Patients

A series of microsphere-conjugated lc probes spanning chromosome 15q11-q13 were hybridized to labeled genomic DNA from patients with large deletions in the PWS/AS region. In some instances deletions were determined by prior analysis (see Methods) (n=8; WJK18, 35, 29, 36, 48, C93-49-PWS, R92-161-PWS, R92-144-PWS), and in other patients the genotypes were unknown however a clinical diagnosis of PWS or AS had been made (n=3; Unknowns 1, 2 and 3).

A map of the PWS region with probes is provided in FIG. 4. In this figure, the chromosomal coordinates for lc probes conjugated to microspheres (highlighted) as well as other microsatellites and genes are indicated. The AS-SRO and PWS-SRO are identified with large stars. Probes conjugated to microspheres are indicated by a vertical dashed line adjoined to a circle representing the level of microsphere which was used for conjugation (1-10). The class I and class II deletion intervals as well as their breakpoint intervals (BP) are depicted. Small stars indicate which probe-conjugated microspheres were used in an initial QMH assay to distinguish class I from class II deletion patients. QMH results illustrate the deleted regions found in patients examined in the present study. Triangles placed on the deletion intervals mark the microsatellite markers tested in each group to define deletion intervals in previous studies (ref). The number of patients found with each type of deletion interval is indicated in parentheses and a black “*” indicates if these individuals are related.

Initial multiplex hybridization reactions to delineate class I from class II deletions in all large deletion patients (n=11) included test probes specific to Chr15Cen, GCP5, D15S11, PWS-SRO and OCA2, and the HOXB1 reference probe. The copy number for all probes was readily distinguishable based on MFI ratios in each patient (Tables 4-4, 4-5 and 4-6). The MFI ratio for deleted regions in this group of patients ranged from 0.44-0.67 and for intact genomic segments ranged from 0.83-1.06 (Tables 4-4, 4-5 and 4-6). Using the results from this initial QMH reaction, subsequent probes were chosen to further narrow each deletion interval. For example in patients WJK-29, WJK-36, and R92-144-PWS, Chr15Cen was intact but, GCP5, D15S11, PWS-SRO and OCA2 were found to be deleted (Tables 4-5 and 4-6). To further delineate these deletion intervals, a QMH probe specific to D15S541 between Chr15Cen and GCP5 was used in a second reaction. Results from this hybridization reaction revealed that WJK-29, WJK-36, and R92-144-PWS are also deleted for D15S541, which accounts for a ˜5.66 Mb deletion interval, and classifies these two patients as class I (Amos-Landgraf et al. 1999). In patients WJK-18, 35, 48, and R92-161-PWS, initial QMH results indicated that Chr15cen and GCP5 were intact, but D15S11, PWS-SRO, and OCA2 were deleted (Tables 4-4 and 4-6). After a second QMH reaction, combined results showed a deletion from AC00659&94501 to OCA2 (˜4.6 Mb), which is considered a class II deletion (Knoll et al. 1990).

QMH results for one patient revealed a non-classical deletion as compared to the typical deleted segment for class I and class II patients (Table 4). In patient C93-49-PWS, probes for D15S63, IC, U41384, AC004600, and OCA2 were present in one copy each, while Chr15Cen, GCP5, AC006596-94501, AC006596-76610, D15S11, and AC004737-13740 were disomic (Table 4-4). The deletion interval spans 3.36 Mb and could represent a sub-class of class II deletions.

### Detection of IC Deletions in PWS Patients

A total of six PWS patients previously found to have IC deletions by microsatellite analysis were used for QMH (R93-001-MOD, R93-002-MOD, R93-013-MOD, R90-035-PWS, R92-166-PWS, and R92-167-PWS) (Tables 4-1, 4-2 and 4-3). For the first family, test probes specific to AC006596-75658, AC004737-13740, D15S63, chr15:22736805, PWS-SRO, U41384, and AC004600, as well as the reference probe, HOXB1, were hybridized to labeled genomic DNA from each family member (Table 3). In R92-166-PWS and R92-167-PWS, a deletion from D15S63 to PWS-SRO was identified (˜93 kb). Previous microsatellite results indicated a deletion at D15S63 and D15S128, so the QMH results extended the deletion found by microsatellite genotyping by ˜70 kb. The unaffected family member (R92-168-PWS) was found normal for all tested loci (Table 4-1).

Another family with a known PWS IC mutation identified previously by Southern hybridization and microsatellite analysis was analyzed by QMH (R93-001-MOD, R93-002-MOD, R93-013-MOD, and R93-014-MOD) (Tables 4-2 and 4-3) (Ohta et al., 1999; Rogan and Knoll, 2003). Initial hybridization reactions included test probes for AC006596-94501, IC, PWS-SRO, and AC004600, in addition to the HOXB1 reference probe. Results showed that all samples analyzed were intact for AC006596-94501 and AC004600, but IC and PWS-SRO were deleted in R93-001-MOD, R93-002-MOD, and R93-013-MOD. Subsequent QMH reactions with test probes circumscribing this deletion interval refined the boundary of the IC deletion from AS-SRO to PWS-SRO (˜50 kb), which extends previous results by ˜45 kb (Buiting et al 1995; Ohta et al. 1999).

QMH was used to analyze the smallest PWS-SRO deletion interval documented to date (˜4.3 kb), R90-035-PWS (Table 4-3) (Ohta et al. 1999). An initial hybridization included probes specific to D 15S63, AS-SRO, IC, PWS-SRO, chr15:22736805, and U41384 in addition to the reference HOXB1 probe (Table 3). MFI ratios indicated all test probes were intact and thus another probe within the region separating the AS-SRO and PWS-SRO, PWS-SRO2, was designed and hybridized to this patient. The PWS-SRO2 probe is located 14.5 kb 3′ (telomeric) of the AS-SRO probe, and 11.7 kb 5′ (centromeric) of the original PWS-SRO QMH probe. An MFI ratio of 0.68 reveals this locus is present in one copy (Table 4-3). The PWS-SRO2 probe was also hybridized to other IC deletion patients in the current study (R92-166-PWS, R93-013-MOD, R93-002-MOD, and R93-001-MOD) and found deleted as expected with MFI ratios ranging from 0.51 to 0.67 (Tables 4-1 and 4-2).

A clinically diagnosed AS patient, Unknown 1, tested as normal by FISH analysis and normal for UPD, however an abnormal SNRPN methylation test was found (Table 4-7). Samples from Unknown 1's mother and father were identified as Unknown 2 and Unknown 3, respectively (Table 4-7). This combination of test results made this patient an ideal candidate for the new assay of the present invention. Using QMH, this unknown patient (Unknown 1), and parents of the patient (Unknown 2 and Unknown 3), were initially screened with Chr15Cen, GCP5, D15S11, PWS-SRO, and OCA2, and the HOXB1 reference probe. Results indicated that all probes were intact, except for PWS-SRO (Table 4). A second QMH analysis focused on the IC region with the AS-SRO and IC probes. MFI ratios indicated that these probes were deleted in the patient and the mother, which constitutes a ˜19 kb deletion around the AS-SRO region (Table 4). This result was confirmed by a third QMH hybridization with the PWS-SRO2 probe, circumscribed by the IC and PWS-SRO probe, and found deleted in both the patient and the mother (Table 4), as well as scFISH analysis with the AS-SRO probe.

### Detection of Proximal Deleted Chromosome 15q

A patient, B2002-00905, with a known proximal chromosome 15q deletion, B2002-00905, was initially characterized by routine cytogenetic analysis and subsequently analyzed using QMH. A multiplex QMH assay included test probes specific to Chr15Cen, D15S541, GCP5, D15S11, D15S63, AC004600, and OCA2 in addition to the reference probe. Results indicated all test probes deleted, accounting for a deletion spanning >6.6 Mb, however no low copy probe sequences 5′ (centromeric) of probe Chr15Cen could be identified due to heterochromatic sequence dense with repeats in this region, and thus the centromeric break in this inversion could not be refined.

### Evaluation of Asynchronous Replication Timing in Maternal Disomy PWS Patient Families

Previous studies (Knoll et al. 1990) examining allele specific replication (ASR) timing within chromosome 15q11-13 revealed paternal early/maternal late replication timing patterns at D15S11 and D15S63. Using QMH probes specific to these two loci, four PWS patients with chromosome 15 maternal disomy (R89-007-PWS, R89-008-PWS, R90-019-PWS, R90-041-PWS) and their family members were tested to see if ASR affected QMH ratios in affected individuals (n=10) (Tables 4-8 and 4-9). The HOXB1 reference probe was used in FISH hybridization studies to verify synchronous replication timing QMH results for the maternal disomic individuals indicate that there is no discernable difference in the MFI ratios as compared to the normal parents. For instance, the MFI ratios for D15S11 and D15S63 in R90-041-PWS were 0.95 and 1.01 (Table 4-9), respectively, which is actually higher as compared to the father, R90-059-PWS, (0.93 and 0.94) but slightly less than the mother, R90-060-PWS, (0.97 and 1.08) (Table 4-8). QMH results for the other two families studied (R90-059-PWS, R90-041-PWS, R89-008-PWS, R89-010-PWS, R89-009-PWS) were synonymous with results for this family.

### Statistical Analysis of QMH Results for Normal and Deleted Loci

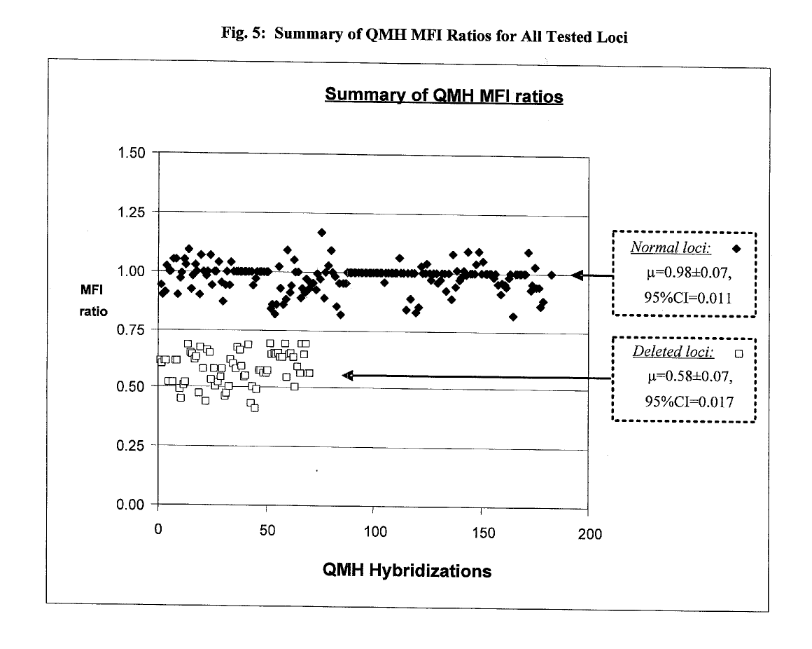

In FIG. 5, MFI ratios are plotted on the y-axis and the number of QMH hybridizations is plotted along the x-axis. There was no overlap in the MFI ratios for normal and deleted probes, hence the copy number at each locus was readily distinguishable. The mean, standard deviation, and 95% confidence intervals for normal and deleted loci are indicated in FIG. 5.

Analysis of the MFI values for all normal loci tested by QMH (n=191) showed a mean of 0.98±0.075 with a 95% confidence level of 0.011 (FIG. 5) and the MFI values for all deleted loci (n=80) have a mean of 0.58±0.072 with a 95% confidence level of 0.016 (FIG. 5). No overlap between the MFI values for normal and deleted loci (n=271) was observed and genotypes were easily distinguishable for all patients which passed the initial PCR quality control step (FIG. 5). It was observed that QMH assays for patient samples stored in methanol:acetic acid for a shorter length of time (˜2-5 years) prior to DNA extraction produced MFI ratios that were consistently closer to 1.0 and 0.5 for normal and deleted loci, respectively, as compared to those stored for longer lengths of time (>5 years). This is likely due to the compromised quality (ie. degraded and sheared condition) of the DNA obtained from fixed cell pellets which have been stored for excessive lengths of time. No patient sample stored for >6 years passed the initial PCR quality control step and thus could not be used for QMH analysis.

### Discussion

This study applies the previously developed quantitative microsphere hybridization (QMH) assay for the detection of copy number changes in the PWS/AS region on chromosome 15q11-13. Using QMH, class I and class II deletions were readily distinguished in a single assay and refined more precisely in a second QMH assay. Smaller deletions within the AS-SRO spanning ˜19 kb were defined as well as deletions as small as 4.3 kb within the PWS-SRO region. One patient exhibited an unusual deletion interval from D15S63 to OCA2 (˜3.36 Mb), which could represent a new class or sub-class of deletions. Hybridization experiments illustrated adequate sensitivity to distinguish one vs. two copies of a genomic sequence for large and small deleted regions. QMH was unable, however, to discern asynchronous replication (ASR) timing patterns in PWS patients arising from maternal disomy. Future studies will include cell synchronization prior to target DNA extraction to further examine this potential application of QMH. The use of distinct spectrally encoded microspheres enabled accurate analysis of up to nine test probes and one reference probe in a single tube.

This study of the PWS/AS region is the first clinical application of the QMH assay and verifies that it is a suitable diagnostic alternative to other copy number determination methods not requiring context-dependent sequence identification. Other techniques to assess copy number in the same region include FISH, Southern hybridization, microarray, aCGH, MLPA, and MAPH. QMH reliably detected copy number differences at a genomic resolution of ≧80 bp, which exceeds the resolution achievable by FISH, Southern hybridization, and aCGH (which is limited by the size and density of cloned probes, and is similar to MAPH and MLPA in probe density. QMH using lc probes does not require either target DNA amplification or the addition of competitor DNA (i.e. Cot-1 DNA) for the suppression of repetitive sequences. The addition of competitor DNA has been shown to compromise the accuracy and reproducibility of genomic hybridization assays (Newkirk et al. 2005), which would not be ideal for a clinical diagnostic test.

QMH was easily adaptable to the PWS/AS region, despite the high density of surrounding repetitive sequences and limited number of low copy regions available for probe design. Assay optimization was minimal (See Methods) and results were consistently reproducible between samples with similar genotypes with negligible differences between MFI ratios (FIG. 5; 0.98±0.075 for intact loci; 0.58±0.072 for deleted loci). With the exception of degraded genomic target DNA, no false positive or false negative results were obtained for samples analyzed in this study. This illustrates the requirement for quality control experiments prior to analysis by QMH to ensure accurate results. The QMH assay of the present invention is an ideal initial diagnostic test for the high-throughput screening of patients with PWS and AS. This test allows for the refinement of PWS/AS deletion intervals using subsequent QMH assays, and FISH could be used to confirm the chromosomal context of deletions detected by QMH.

The microsphere hybridization platform may be more appropriate in some diagnostic situations instead of FISH, array comparative genomic hybridization (aCGH), Southern analysis, multiplex amplifiable probe hybridization (MAPH), multiplex ligation probe hybridization (MLPA) and quantitative PCR (qPCR). The microsphere assay of the present invention improves resolution, increases signal to noise ratio, and is easily amenable to multiplexing, making it a high throughput approach. The ability to multiplex probes conserves available patient material and decreases time needed to make diagnoses. Further, contamination of heterologous genomic sequences was shown to not impact the hybridization efficiency in the assay.

In the QMH assay, differences in copy number are detected with a genomic resolution of as little as 100 bp at a level of less than 5 genomic equivalents per hybridization reaction. The level of resolution achieved with conventional FISH and Southern hybridization is ˜20 kb to 650 kb. With aCGH, the resolution is limited to the size and density of cloned probes and the variability in levels of fluorescence signal can affect accuracy of interpretation. MAPH has a resolution of approximately 50 kb, and MLPA, while it has higher resolution than other techniques, is sensitive to single nucleotide polymorphisms at or near ligation sites which can lead to false negative results. Like qPCR, care must be taken to prevent contamination, and neither technique is easily multiplexed.

With genomic arrays of lc microsphere-conjugated suspension hybridization probes, it should be feasible to rapidly delineate the extent of chromosomal abnormalities that alter normal copy number. Hybridization of a dense set of lc probes that span a chromosomal region could be used to initially screen for the boundaries of aneusomic domains. Subsequent FISH studies could then provide a chromosomal context for these genetic alterations. This invention lends itself to high throughput applications since, in theory, 3840 different products could be assayed on a single high density microtiter plate using our current set of 10 spectrally distinct encoded beads in each well. With current instrumentation, assuming a minimum probe resolution of 20 kb, a single plate could provide high-resolution copy number determination for a 20 Mb domain within 90 minutes. Based on the demonstrated sensitivity and accuracy of the method, microsphere suspension hybridization should be naturally extensible to other applications, such as the detection and quantification of low copy or rare mRNA sequences (˜5 genome equivalents per hybridization reaction) in cDNA.

### References for Example 1

The following reference materials are hereby incorporated by reference.
Armour J A L, Sismani C, Patsalis P C, Cross G. 2000. Measurement of locus copy number by hybridisation with amplifiable probes. Nucleic Acids Res. 28 (2):605-609.
Bagwell C B, Baker D, Whetstone S, Munson M, Hitchcox S, Ault K A, Lovett E J. 1989. A simple and rapid method for determining the linearity of a flow cytometer amplification system. Cytometry 10 (6):689-94.
Brown R D, Zarbo R J, Linden M D, Torres F X, Nakleh R E, Schultz D, Mackowiak P G. 1994. Two-color multiparametric method for flow cytometric DNA analysis. Standardization of spectral compensation. American Journal of Clinical Pathology 101 (5):630-637.
Coder D M, Redelman D, Vogt R F. 1994. Computing the central location of immunofluorescence distributions: logarithmic data transformations are not always appropriate. Cytometry 18 (2):75-8.
Dunbar S, Godbout R, Newkirk H, Hetzel J. 2003. Microsphere suspension array technology for SNP detection in cattle. IEEE Eng Med Biol Mag 22 (4):158-62.
Earley M, Vogt R J, Shapiro H, Mandy F, Kellar K, Bellisario R, Pass K, Marti G, Stewart C, Hannon W. 2002. Report from a workshop on multianalyte microsphere. Cytometry 50 (5):239-42.
Fulton R J, McDade R L, Smith P L, Kienker L J, Kettman J R, Jr. 1997. Advanced multiplexed analysis with the FlowMetrix system. Clin Chem 43 (9):1749-56.
Hadd A G, Laosinchai-Wolf W, Novak C R, Badgett M R, Isgur L A, Goldrick M, Walkerpeach C R. 2004. Microsphere bead arrays and sequence validation of 5/7/9T genotypes for multiplex screening of cystic fibrosis polymorphisms. J Mol Diagn 6 (4):348-55.
Inoue K, Dewar K, Katsanis N, Reiter L T, Lander E S, Devon K L, Wyman D W, Lupski J R, Birren B. 2001. The 1.4-Mb CMT1A Duplication/HNPP Deletion Genomic Region Reveals Unique Genome Architectural Features and Provides Insights into the Recent Evolution of New Genes. Genome Research 11 (6):1018-1033.
Knoll J H, Lichter P. 1994. In situ hybridization to metaphase chromosomes and interphase nuclei. In Dracopoli N C, Haines J L, Korf B R, Moir D T, Morton C C, Seidman C E, Seidman J G, Smith D R (eds): “Current protocols in Human Genetics Volume 1” Unit 4.3, Green-Wiley, New York. (Revised 2005, in press).
Knoll J H, Rogan P K. 2003. Sequence-based, in situ detection of chromosomal abnormalities at high resolution. American Journal of Medical Genetics 121 (3):245-257.
Knoll J H M, Rogan P K. 2004. Single Copy Genomic Hybridization Probes and Method of Generating Same. U.S. Pat. No. 6,828,097.
Lewin B. 1980. DNA sequence organization: nonrepetitive and repetitive DNA. In “Gene Expression 2—Eucaryotic Chromosomes”, Chapter 18, pp 503-530, 2^{nd} edition, Wiley-Interscience Publication.
Lupski J R, Oca-Luna R M d, Slaugenhaupt S, Pentao L, Guzzetta V, Trask B J, Saucedo-Cardenas O, Barker D F, Killian J M, Garcia C A et al. 1991. DNA duplication associated with Charcot-Marie-Tooth disease type 1A. Cell 66 (2):219-32.
Oostlander A E, Meijer G A, Ylstra B. 2004. Microarray-based comparative genomic hybridization and its applications in human genetics. Clin Genet 66 (6):488-95.
Rockenbauer E, Petersen K, Vogel U, Bolund L, Kolvraa S, Nielsen K V, Nexo B A. 2005. SNP genotyping using microsphere-linked PNA and flow cytometric detection. Cytometry A 64 (2):80-6.
Rogan P K, Cazcarro P M, Knoll J H. 2001. Sequence-based design of single-copy genomic DNA probes for fluorescence in situ hybridization. Genome Res 11 (6): 1086-94.
Schouten J P, McElgunn C J, Waaijer R, Zwijnenburg D, Diepvens F, Pals G. 2002. Relative quantification of 40 nucleic acid sequences by multiplex ligation-dependent probe amplification. Nucleic Acids Res 30 (12):e57.
Sekar M, Bloch W, John P S. 2005. Comparative study of sequence-dependent hybridization kinetics in solution and on microspheres. Nucleic Acids Res. 33 (1):366-75.
Shaffer, L G, Bejjani, B A. 2004. A cytogeneticist's perspective on genomic microarrays. Hum Reprod Update. 10 (3):221-26.
Sinclair P, Nacheva E, Leversha M, Telford N, Chang J, Reid A, Bench A, Champion K, Huntly B, Green A. 2000. Large deletions at the t(9;22) breakpoint are common and may identify a poor-prognosis subgroup of patients with chronic myeloid leukemia. Blood 95 (3):738-43.
Southern E M. 1975. Detection of specific sequences among DNA fragments separated by gel electrophoresis. J Mol Biol 98:503-518.
Vignali D. 2000. Multiplexed particle-based flow cytometric assays. J Immunol Methods. 243 (1-2):243-55.
White S J, Breuning M H, den Dunnen J T. 2004. Detecting copy number changes in genomic DNA: MAPH and MLPA. Methods Cell Biol. 75:751-768.

### References for Example 2

The following reference materials are hereby incorporated by reference.
Amos-Landgraf J M, Ji Y, Gottlieb W, Depinet T, Wandstrat A E, Cassidy S B, Driscoll D J, Rogan P K, Schwartz S, Nicholls R D (1999) Chromosome breakage in the Prader-Willi and Angelman syndromes involves recombination between large, transcribed repeats at proximal and distal breakpoints. Am J Hum Genet 65:370-386.
Bejjani B A, Shaffer L G (2004) A cytogeneticist's perspective on genomic microarrays. Hum Reprod Update 10:221-226
Bittel D C, Butler M G (2005) Prader-Willi syndrome: clinical genetics, cytogenetics and molecular biology. Expert Reviews in Molecular Medicine 7:1-20.
Bittel D C, Kibiryeva N, Talebizadeh Z, Butler M G (2003) Microarray analysis of gene/transcript expression in Prader-Willi syndrome: deletion versus UPD. J Med Genet 40:568-574.
Bittel D C, Kibiryeva N, Talebizadeh Z, Driscoll D J, Butler M G (2005) Microarray analysis of gene/transcript expression in Angelman syndrome: deletion versus UPD. Genomics 85:85-91.
Buiting K, Saitoh S, Gross S, Dittrich B, Schwartz S, Nicholls R D, Horsthemke B (1995) Inherited microdeletions in the Angelman and Prader-Willi syndromes define an imprinting centre on human chromosome 15. Nature Genetics 9:395-400
Knoll J, Rogan P (2004) Single Copy Genomic Hybridization Probes and Method of Generating Same. U.S. Pat. No. 6,828,097
Knoll J H, Rogan P K (2003) Sequence-based, in situ detection of chromosomal abnormalities at high resolution. American Journal of Medical Genetics 121:245-257
Knoll J H M, Nicholls R D, Magenis R E, Glatt K, J. M. Graham J, Kaplan L, Lalande M (1990) Angelman syndrome: three molecular classes identified with chromosome 15q11q13-specific DNA markers. Am J Hum Genet 47:149-155
Mapendano C K, Kishino T, Miyazaki K, Kondo S, Yoshiura K-I, Hishikawa Y, Koji T, Niikawa N, Ohta T (2006) Expression of the Snurf-Snrpn IC transcript in the oocyte and its putative role in the imprinting establishment of the mouse 7C imprinting domain. J Hum Genet 51:236-243
Newkirk H, Knoll J H M, Rogan P (2005) Distortion of quantitative genomic and expression hybridization by Cot-1 DNA: mitigation of this effect. Nucleic Acids Research 33:e191
Newkirk H, Miralles M, Rogan P, Knoll J H M (2006) Determination of genomic copy number with quantitative microsphere hybridization. Human Mutation 27:376-386
Nygren A O H, Ameziane N, Duarte H M B, Vijzelaar R N C P, Waisfisz Q, Hess C J, Schouten J P, Errami A (2005) Methylation-specific MLPA (MS-MLPA): simultaneous detection of CpG methylation and copy number changes of up to 40 sequences. Nucleic Acids Research 33:e128
Ohta T, Gray T A, Rogan P K, Buiting K, Gabriel J M, Saitoh S, Muralidhar B, Bilienska B, Krajewska-Walasek M, Driscoll D J, Horsthemke B, Butler M G, Nicholls R D (1999) Imprinting-mutation mechanisms in Prader-Willi syndrome. Am J Hum Genet 64:397-413
Sahoo T, Shaw C A, Young A S, Whitehouse N L, Schroer R J, Stevenson R E, Beaudet A L (2005) Array-based comparative genomic hybridization analysis of recurrent chromosome 15q rearrangements. Am J Med Genet 139A: 106-113
Schouten J P, McElgunn C J, Waaijer R, Zwijnenburg D, Diepvens F, Pals G (2002) Relative quantification of 40 nucleic acid sequences by multiplex ligation-dependent probe amplification. Nucleic Acids Res 30:e57

TABLE 1
Characteristics of Hybridization Probes

| | | Length | Hyb | Chromo- | |
|---|---|---|---|---|---|
| Gene | Probe | (bp) | temp | some | Coordinates (Hg 12) |
| HOXB1 | HoxB1a | 2286 | 51 | 17 | 43965662-43967948 |
| | HoxB1b | 1343 | 45 | 17 | 43962053-43963396 |
| | HoxB1c | 102 | 50 | 17 | 43964237-43964330 |
| ABL1 | 16-1a | 2304 | 45 | 9 | 130623551-130625854 |
| | 16-1b | 100 | 50 | 9 | 130624671-130624771 |
| | 16-2a | 1381 | 48 | 9 | 130627353-130628735 |
| | 16-2b | 101 | 50 | 9 | 130627353-130627454 |
| TEKT3 | TEKT3 | 98 | 50 | 17 | 15149108-15149206 |
| PMP22 | PMP22 | 101 | 50 | 17 | 15073475-15073576 |

TABLE 2
Flow Cytometric Detection of Chromosome Abnormalities

| | | | Mean Intensities | | | |
|---|---|---|---|---|---|---|
| | | | | Reference | | |
| Sample | Cytogenetic findings | Test Probe | test probe | probe | Ratio* | Genotype |
| 47 2 | t(9; 22)(q34; q11.2).ish der(9)(5′ABL+) | 16-1b | 48.72 | 50.94 | 0.96 | wt |
| 38.00 | t(9; 22)(q34; q11.2).ish der(9)(5′ABL+) | 16-1b | 39.22 | 40.22 | 0.98 | wt |
| 195.00 | t(9; 22)(q34; q11.2).ish der(9)(5′ABL+) | 16-1b | 1240.47 | 1179.37 | 1.05 | wt |
| 195 3 | ibid | 16-1b | 1121.98 | 1132.11 | 0.99 | wt |
| CMT1A-1 | nuc ish 17p12(PMP22x3) | 16-1b | 41.04 | 44.74 | 0.92 | wt |
| 177.00 | t(9; 22)(q34; q11.2).ish der(9)(ABL+) | 16-1b | 182.41 | 173.86 | 1.05 | wt |
| 177 3 | ibid | 16-1b | 162.15 | 161.31 | 1.01 | wt |
| 86.00 | t(9; 22)(q34; q11.2).ish der(9)(5′ABL+) | 16-1b | 107.40 | 104.20 | 1.03 | wt |
| 86 3 | ibid | 16-1b | 124.63 | 114.80 | 1.09 | wt |
| 124.00 | t(9; 22)(q34; q11.2).ish der(9)(5′ABL+) | 16-1b | 721.63 | 678.48 | 1.06 | wt |
| 77.00 | t(9; 22)(q34; q11.2).ish der(9)(5′ABL+) | 16-1b | 122.30 | 119.63 | 1.02 | wt |
| 47.00 | t(9; 22)(q34; q11.2).ish der(9)(5′ABL+) | 16-2b | 44.31 | 51.74 | 0.86 | wt |
| 38.00 | t(9; 22)(q34; q11.2).ish der(9)(5′ABL+) | 16-2b | 40.22 | 44.99 | 0.89 | wt |
| 47.00 | t(9; 22)(q34; q11.2).ish der(9)(5′ABL+) | 16-2b | 27.41 | 26.77 | 1.02 | wt |
| 86.00 | t(9; 22)(q34; q11.2).ish der(9)(5′ABL+) | PMP22 | 84.99 | 82.70 | 1.03 | wt |
| 81*** | t(9; 22)(q34; q11.2).ish der(9)(5′ABL−) | PMP22 | 203.47 | 202.15 | 1.01 | wt |
| 86*** | t(9; 22)(q34; q11.2).ish der(9)(ABL+) | PMP22 | 84.99 | 82.70 | 1.03 | wt |
| 86.00 | ibid | PMP22 | 84.99 | 82.70 | 1.03 | wt |
| 81.00 | t(9; 22)(q34; q11.2).ish der(9)(5′ABL−) | PMP22 | 203.47 | 202.15 | 1.01 | wt |
| 81 3 | ibid | PMP22 | 190.66 | 194.03 | 0.98 | wt |
| 86.00 | t(9; 22)(q34; q11.2).ish der(9)(ABL+) | TEKT3 | 97.22 | 99.60 | 0.98 | wt |
| 33.00 | t(9; 22)(q34; q11.2).ish der(9)(5′ABL−) | 16-1b | 12.87 | 26.35 | 0.49 | del |
| 33.00 | ibid | 16-1b | 58.76 | 91.19 | 0.64 | del |
| 81.00 | t(9; 22)(q34; q11.2).ish der(9)(5′ABL−) | 16-1b | 28.02 | 41.48 | 0.68 | del |
| 33.00 | t(9; 22)(q34; q11.2).ish der(9)(5′ABL−) | 16-1b | 12.87 | 26.35 | 0.49 | del |
| 46.00 | t(9; 22)(q34; q11.2).ish der(9)(5′ABL−) | 16-1b | 82.77 | 142.28 | 0.58 | del |
| 33 2 | t(9; 22)(q34; q11.2).ish der(9)(5′ABL−) | 16-2b | 69.43 | 103.90 | 0.67 | del |
| 81.00 | t(9; 22)(q34; q11.2).ish der(9)(5′ABL−) | 16-2b | 46.78 | 91.19 | 0.51 | del |
| 33.00 | t(9; 22)(q34; q11.2).ish der(9)(5′ABL−) | 16-2b | 46.78 | 88.81 | 0.53 | del |
| GM10186 | 47, XY, +9.ish 9p11q11(D9Z1x3) | 16-1b | 75.23 | 59.16 | 1.27 | dup |
| GM06074 | 46, XX, rec(9)dup(9)inv ins(9)(q22.1; q34.3q34.1) | 16-1b | 97.95 | 77.63 | 1.26 | dup |
| GM10186³ | 47, XY, +9.ish 9p11q11(D9Z1x3) | 16-1b | 104.00 | 74.37 | 1.40 | dup |
| GM09286³ | 47, XY, +9.ish 9p11q11(D9Z1x3) | 16-1b | 65.10 | 48.72 | 1.34 | dup |
| GM09286 | 47, XY, +9.ish 9p11q11(D9Z1x3) | 16-1b | 55.47 | 37.93 | 1.46 | dup |
| GM10186 | 47, XY, +9.ish 9p11q11(D9Z1x3) | 16-1b | 57.94 | 35.61 | 1.63 | dup |
| CMT1A-1 | nuc ish 17p12(PMP22x3) | PMP22 | 965.98 | 564.61 | 1.71 | dup |
| CMT1A-2 | nuc ish 17p12(PMP22x3) | PMP22 | 556.73 | 342.57 | 1.63 | dup |
| CMT1A-3 | nuc ish 17p12(PMP22x3) | PMP22 | 964.76 | 521.22 | 1.85 | dup |
| CMT1A-4 | nuc ish 17p12(PMP22x3) | PMP22 | 626.91 | 356.77 | 1.76 | dup |
| CMT1A-1*** | nuc ish 17p12(PMP22x3) | PMP22 | 196.31 | 121.56 | 1.61 | dup |
| CMT1A-2*** | nuc ish 17p12(PMP22x3) | PMP22 | 159.39 | 110.60 | 1.44 | dup |
| CMT1A-3*** | nuc ish 17p12(PMP22x3) | PMP22 | 90.11 | 64.04 | 1.41 | dup |
| GM12214 | D17S122x3⁴ | PMP22 | 869.93 | 530.49 | 1.64 | dup |
| CMT1A-1 | nuc ish 17p12(PMP22x3) | TEKT3 | 104.09 | 74.17 | 1.40 | dup |
| CMT1A-1-1** | nuc ish 17p12(PMP22x3) | TEKT3 | 3313.34 | 1990.96 | 1.66 | dup |
| CMT1A-1³ | ibid | TEKT3 | 125.56 | 79.62 | 1.58 | dup |
| CMT1A-1³ | ibid | TEKT3 | 70.65 | 48.21 | 1.47 | dup |
| CMT1A-1³ | ibid | TEKT3 | 107.43 | 76.43 | 1.41 | dup |
| CMT1A-2 | nuc ish 17p12(PMP22x3) | TEKT3 | 70.65 | 48.21 | 1.47 | dup |
| CMT1A-2³ | ibid | TEKT3 | 122.33 | 86.02 | 1.42 | dup |
| CMT1A-2³ | ibid | TEKT3 | 104.09 | 74.17 | 1.40 | dup |
| CMT1A-2³ | ibid | TEKT3 | 107.62 | 74.36 | 1.45 | dup |
| CMT1A-3 | nuc ish 17p12(PMP22x3) | TEKT3 | 122.33 | 86.02 | 1.42 | dup |
| CMT1A-4 | nuc ish 17p12(PMP22x3) | TEKT3 | 107.43 | 76.43 | 1.41 | dup |
| GM12214 | D17S122x3⁴ | TEKT3 | 181.33 | 131.19 | 1.38 | dup |

*Ratio of geometric mean fluorescence
**HOXB1d used as reference probe
***16-1b used as reference probe
****HOXB1a used as reference probe
wt: wild type; del: deletion; dup: duplication
¹HOXB1c probe included in each hybridization as reference for two copies unless otherwise noted
²Samples run in blind study
³Samples run in reproducibility experiments for hybridizations performed on different days
⁴Duplication reported at locus by Lupski et al, 1991

TABLE 3
QMH probes utilized in this study

| | Chromosomal location | |
|---|---|---|
| Probe | (May 2004)* | Copy Number |
| Chr15Cen | chr15:19412079 | sc |
| D15S541 | chr15:20360015 | sc |
| GCP5 | chr15:20402365 | sc |
| AC006596-94501 | chr15:21418753 | sc |
| AC006596-76610 | chr15:21436280 | sc |
| AC006596-75658 | chr15:21437980 | sc |
| D15S11 | chr15:21625540 | sc |
| 4737-13740 | chr15:22569559 | sc |
| D15S63 | chr15:22658040 | lc |
| AS-SRO | chr15:22717169 | sc |
| IC | chr15:22736450 | lc |
| chr15:22736805 | chr15:22736805 | lc |
| PWS-SRO2 | chr15:22751461 | sc |
| PWS-SRO | chr15:22763150 | sc |
| U41384 | chr15:22751151 | sc |
| SNRPN-5′ | chr15:22736850 | sc |
| AC004600 | chr15:23123664 | sc |
| OCA2 | chr15:26017960 | sc |
| HOXB1 | chr17:43963520 | lc |

*Coordinate indicates 5′ end of QMH probe. The length of each probe is 80 nucleotides.

TABLE 4-1
PWS-IC Deletion

| | R92-166-PWS | | R92-167-PWS | | R92-168-PWS | |
|---|---|---|---|---|---|---|
| Probes | MFI | MFI RATIO | MFI | MFI RATIO | MFI | MFI RATIO |
| Chr15Cen | | | | | | |
| D15S541 | | | | | | |
| GCP5 | | | | | | |
| AC006596-94501 | | | | | | |
| AC006596-76610 | | | | | | |
| AC006596-75658 | 877.68 | 0.92 | 775.26 | 0.98 | 447.16 | 1.03 |
| D15S11 | | | | | | |
| 4737-13740 | 867.25 | 0.9 | 752.98 | 0.91 | 442.1 | 1.02 |
| D15S63 | 581.17 | 0.61 | 387.25 | 0.49 | 407.53 | 0.94 |
| AS-SRO | | | | | | |
| chr15: 22736805 | 554.11 | 0.61 | 361.49 | 0.45 | | |
| IC | | | | | | |
| PWS-SRO2 | 344.29 | 0.67 | 397.53 | 0.66 | | |
| PWS-SRO | 554.11 | 0.61 | 400.98 | 0.51 | 436.9 | 1 |
| U41384 | 862.51 | 0.95 | 540.17 | 0.99 | 446.47 | 1.03 |
| AC004600 | 812.56 | 0.9 | 766.7 | 0.973 | 454.03 | 1.05 |
| OCA2 | | | | | | |
| HOXB1 | 953.07 | 1 | 786.03 | 1 | 433.77 | 1 |
| HoxB1 | 904.58 | 1 | 543.66 | | | |
| HoxB1 | 953.07 | 1 | | | | |
| HOXB1 | 511.56 | | | | | |
| HOXB1 | | | | | | |

TABLE 4-2
PWS-IC Deletion

| | R93-001-MOD | | R93-002-MOD | | R93-013-MOD | |
|---|---|---|---|---|---|---|
| Probes | MFI | MFI RATIO | MFI | MFI RATIO | MFI | MFI RATIO |
| Chr15Cen | | | | | 608.86 | 1.04 |
| D15S541 | | | | | | 1 |
| GCP5 | | | | | 591.17 | 1.07 |
| AC006596-94501 | | | | | 623.74 | |
| AC006596-76610 | | | | | 637.56 | 1 |
| AC006596-75658 | | | | | 650.75 | 1.09 |
| D15S11 | | | | | 550.78 | |
| 4737-13740 | | | | | 629.24 | 0.94 |
| D15S63 | 349.83 | 0.93 | 218.07 | 0.89 | 685.65 | 1.1 |
| AS-SRO | 279.54 | 0.48 | 477.62 | 0.5 | 287.44 | 0.54 |
| chr15: 22736805 | 1177 | 0.52 | 558.24 | 0.6 | 576.46 | 0.62 |
| IC | | | 560.08 | 0.61 | 668.84 | 0.56 |
| PWS-SRO2 | 168.4 | 0.51 | 140.18 | 0.58 | 474.64 | 0.67 |
| PWS-SRO | 611.92 | 0.52 | 560.08 | 0.61 | 576.46 | 0.62 |
| U41384 | 353.57 | 0.99 | 628.65 | 1.01 | 656.62 | 1.03 |
| AC004600 | | | | | 642.6 | 1.01 |
| OCA2 | | | | | 583.66 | 1 |
| HOXB1 | 355.59 | 1 | 925.21 | 1 | 706.99 | 1 |
| HoxB1 | 611.92 | 1 | 617.84 | 1 | 919.25 | 1 |
| HoxB1 | 1177 | 1 | 244.96 | 1 | 583.5 | 1 |
| HOXB1 | 332.33 | 1 | 240.36 | | 1171.82 | 1 |
| HOXB1 | | | | | 623.05 | 1 |

TABLE 4-3
PWS-IC Deletion

| | R93-014-MOD | | R90-035-PWS | |
|---|---|---|---|---|
| Probes | MFI | MFI RATIO | MFI | MFI RATIO |
| Chr15Cen | | | | |
| D15S541 | | | | |
| GCP5 | | | | |
| AC006596-94501 | 1040.10 | 1.04 | | |
| AC006596-76610 | 551.24 | 1 | | |
| AC006596-75658 | 589.73 | 1.07 | | |
| D15S11 | | | | |
| 4737-13740 | 548.96 | 1 | | |
| D15S63 | 1099.32 | 1.09 | 2218.29 | 0.96 |
| AS-SRO | 550.84 | 0.94 | 1369.26 | 0.91 |
| chr15:22736805 | 1002.80 | 1.01 | 539.61 | 0.94 |
| IC | | | 2282.87 | 0.98 |
| PWS-SRO2 | | | 420.72 | 0.68 |
| PWS-SRO | 550.84 | 0.94 | 2282.87 | 0.98 |
| U41384 | 601.70 | 1.09 | 2156.66 | 0.93 |
| AC004600 | 1142.21 | 1.05 | | |
| OCA2 | | | | |
| HOXB1 | | | 571.37 | 1 |
| HoxB1 | 585.35 | 1 | 2318.37 | 1 |
| HoxB1 | 585.35 | 1 | 2318.87 | 1 |
| HOXB1 | 971.38 | 1 | 617.43 | 1 |
| HOXB1 | 1004.81 | 1 | | |

TABLE 4-4

| | C93-49-PWS | | WJK-18 | | WJK-35 | |
|---|---|---|---|---|---|---|
| Probes | MFI | MFI RATIO | MFI | MFI RATIO | MFI | MFI RATIO |
| Chr15Cen | 1393.07 | 0.98 | 2253 | 1.02 | 2132.08 | 0.93 |
| D15S541 | | | 1410.88 | 0.85 | 403.88 | 0.95 |
| GCP5 | 1341.11 | 0.94 | 4997.03 | 0.84 | 1614.86 | 0.89 |
| AC006596-94501 | 1743.21 | 0.98 | 1923.16 | 0.47 | 2480.87 | 0.66 |
| AC006596-76610 | | | | | | |
| AC006596-75658 | 7219.48 | 1 | | | | |
| D15S11 | 1432.32 | 1 | 2743.42 | 0.67 | 2436.3 | 0.65 |
| 4737-13740 | 1270.05 | 1.06 | | | | |
| D15S63 | 1226.86 | 0.68 | | | | |
| AS-SRO | | | | | | |
| chr15: 22736805 | | | | | | |
| IC | 902 | 0.65 | | | | |
| PWS-SRO2 | | | | | | |
| PWS-SRO | | | 1923.16 | 0.58 | 2022.45 | 0.53 |
| U41384 | 792.40 | 0.64 | | | | |
| AC004600 | 1290.12 | 0.62 | | | | |
| OCA2 | 864.81 | 0.63 | 1798.12 | 0.44 | 2187.31 | 0.58 |
| HOXB1 | 1299.39 | 1 | 2219.26 | 1 | 2301.9 | 1 |
| HoxB1 | 1187.70 | 1 | 4095.52 | 1 | 3773.98 | 1 |
| HoxB1 | 1418.39 | 1 | 5941.3 | 1 | 1808 | 1 |
| HOXB1 | 1782.99 | 1 | | | 422.31 | 1 |
| HOXB1 | | | | | | |

TABLE 4-5

| | WJK-29 | | WJK-36 | |
|---|---|---|---|---|
| Probes | MFI | MFI RATIO | MFI | MFI RATIO |
| Chr15Cen | 1877.33 | 0.83 | 1956.96 | 0.85 |
| D15S541 | 644.15 | 0.50 | 403.88 | 0.47 |
| GCP5 | 1176.46 | 0.52 | 1164.56 | 0.50 |
| AC006596-94501 | | | | |
| AC006596-76610 | | | | |
| AC006596-75658 | | | | |
| D15S11 | 1215.87 | 0.54 | 1429.98 | 0.62 |
| 4737-13740 | | | | |
| D15S63 | | | | |
| AS-SRO | | | | |
| chr15:22736805 | | | | |
| IC | | | | |
| PWS-SRO2 | | | | |
| PWS-SRO | 1306.65 | 0.58 | 1394.38 | 0.60 |
| U41384 | | | | |
| AC004600 | | | | |
| OCA2 | 1035.14 | 0.46 | 1335.82 | 0.58 |
| HOXB1 | 2265.51 | 1 | 2308.24 | 1 |
| HoxB1 | | | | |
| HoxB1 | | | | |
| HOXB1 | | | | |
| HOXB1 | | | | |

TABLE 4-6

| | WJK-48 | | R92-161-PWS | | R92-144-PWS | |
|---|---|---|---|---|---|---|
| Probes | MFI | MFI RATIO | MFI | MFI RATIO | MFI | MFI RATIO |
| Chr15Cen | 2922.74 | 0.86 | 1283.70 | 0.95 | 1299.39 | 1 |
| D15S541 | 2786.61 | 0.82 | | | 186.46 | 0.48 |
| GCP5 | 698.20 | 1.03 | 1410.51 | 1.04 | 896.12 | 0.69 |
| AC006596-94501 | 2328.13 | 0.67 | 461.24 | 0.55 | | |
| AC006596-76610 | | | | | | |
| AC006596-75658 | | | | | | |
| D15S11 | 2254.42 | 0.66 | 1337.82 | 0.68 | 1831.17 | 0.64 |
| 4737-13740 | | | | | 1845.7 | 0.65 |
| D15S63 | | | | | 1816.35 | 0.64 |
| AS-SRO | | | | | 325.03 | 0.55 |
| chr15: 22736805 | | | | | 379.93 | 0.64 |
| IC | | | 825.57 | 0.43 | 902 | 0.65 |
| PWS-SRO2 | | | | | | |
| PWS-SRO | 2038.45 | 0.59 | 825.57 | 0.50 | 902 | 0.63 |
| U41384 | | | | | | |
| AC004600 | | | 791.49 | 0.41 | | |
| OCA2 | 1832.27 | 0.54 | 803.48 | 0.49 | 864.81 | 0.63 |
| HOXB1 | 3408.41 | 1 | | | 2851.18 | 1 |
| HoxB1 | 3081.26 | 1 | 1941.53 | 1 | 1382.82 | 1 |
| HoxB1 | 676.06 | 1 | 1650.98 | 1 | 391.16 | 1 |
| HOXB1 | | | | | 586.61 | |
| HOXB1 | | | | | | |

TABLE 4-7

| | Unknown 1 | | Unknown 2 | | Unknown 3 | |
|---|---|---|---|---|---|---|
| Probes | MFI | MFI RATIO | MFI | MFI RATIO | MFI | MFI RATIO |
| Chr15Cen | 1651.72 | 0.95 | | | | |
| D15S541 | | | | | | |
| GCP5 | 1677.97 | 0.97 | | | | |
| AC006596-94501 | | | | | | |
| AC006596-76610 | | | | | | |
| AC006596-75658 | | | | | | |
| D15S11 | 1828.09 | 1.05 | | | | |
| 4737-13740 | | | | | | |
| D15S63 | 1460.10 | 0.94 | 1109.36 | 0.94 | 903.29 | 0.87 |
| AS-SRO | 1347.48 | 0.57 | 661.00 | 0.56 | 853.71 | 0.82 |
| chr15: 22736805 | | | | | | |
| IC | 668.84 | 0.57 | | | | |
| PWS-SRO2 | 218.17 | 0.55 | 273.47 | 0.56 | 581.53 | 0.99 |
| PWS-SRO | 668.84 | 0.56 | 665.00 | 0.57 | 895.97 | 0.86 |
| U41384 | 1564.10 | 1 | 1123.51 | 0.96 | 1011.98 | 0.97 |
| AC004600 | | | | | | |
| OCA2 | 1677.21 | 0.97 | | | | |
| HOXB1 | 1179.61 | 1 | 1175 | 1 | 1040.93 | 1 |
| HoxB1 | 1844.28 | 1.06 | 488.95 | 1 | 588.24 | 1 |
| HoxB1 | 1548.64 | 1 | | | | |
| HOXB1 | 392.37 | 1 | | | | |

TABLE 4-8

| | R90-019-PWS* | | R90-021-PWS | | R90-20-PWS | | R90-060-PWS | | R90-059-PWS | |
|---|---|---|---|---|---|---|---|---|---|---|
| Probes | MFI | MFI RATIO | MFI | MFI RATIO | MFI | MFI RATIO | MFI | MFI RATIO | MFI | MFI RATIO |
| Chr15Cen | | | | | | | | | | |
| D15S541 | | | | | | | | | | |
| GCP5 | | | | | | | | | | |
| AC006596-94501 | | | | | | | | | | |
| AC006596-76610 | | | | | | | | | | |
| AC006596-75658 | | | | | | | | | | |
| D15S11 | 495.57 | 0.89 | 407.11 | 0.93 | 338.63 | 0.91 | 764.84 | 0.97 | 267.56 | 0.93 |
| 4737-13740 | | | | | | | | | | |
| D15S63 | 515.66 | 0.92 | 433.86 | 1 | 332.27 | 0.89 | 853.61 | 1.08 | 271.85 | 0.94 |
| AS-SRO | | | | | | | | | | |
| chr15: 22736805 | | | | | | | | | | |
| IC | | | | | | | | | | |
| PWS-SRO2 | | | | | | | | | | |
| PWS-SRO | | | | | | | | | | |
| U41384 | | | | | | | | | | |
| AC004600 | | | | | | | | | | |
| OCA2 | | | | | | | | | | |
| HOXB1 | 556.67 | 1 | 433.19 | 1 | 369.99 | 1 | 786.74 | 1 | 286.99 | 1 |
| HoxB1 | | | | | | | | | | |
| HoxB1 | | | | | | | | | | |
| HOXB1 | | | | | | | | | | |

TABLE 4-9

| | R90-041-PWS* | | R89-008-PWS* | | R89-007-PWS* | | R89-010-PWS | | R89-009-PWS | |
|---|---|---|---|---|---|---|---|---|---|---|
| Probes | MFI | MFI RATIO | MFI | MFI RATIO | MFI | MFI RATIO | MFI | MFI RATIO | MFI | MFI RATIO |
| Chr15Cen | | | | | | | | | | |
| D15S541 | | | | | | | | | | |
| GCP5 | | | 585.89 | 0.84 | 440.17 | 0.86 | 533.35 | 0.82 | 398.31 | 0.86 |
| AC006596-94501 | | | | | | | | | | |
| AC006596-76610 | | | | | | | | | | |
| AC006596-75658 | | | | | | | | | | |
| D15S11 | 457.63 | 0.95 | 476.84 | 0.95 | 351.34 | 0.92 | 314.43 | 0.99 | 534.92 | 0.97 |
| 4737-13740 | | | | | | | | | | |
| D15S63 | 484.20 | 1.01 | 476.12 | 0.95 | 346.58 | 0.91 | 305.53 | 0.96 | 522.52 | 0.95 |
| AS-SRO | | | | | | | | | | |
| chr15: 22736805 | | | | | | | | | | |
| IC | | | | | | | | | | |
| PWS-SRO2 | | | | | | | | | | |
| PWS-SRO | | | | | | | | | | |
| U41384 | | | | | | | | | | |
| AC004600 | | | | | | | | | | |
| OCA2 | | | 671.45 | 0.97 | 498.49 | 0.98 | 660.90 | 1.01 | 459.97 | 0.99 |
| HOXB1 | 479.32 | 1 | 501.16 | 1 | 379.95 | 1 | 317.69 | 1 | 551.04 | 1 |
| HoxB1 | | | | | | | | | | |
| HoxB1 | | | | | | | | | | |
| HOXB1 | | | | | | | | | | |

TABLE 5
Average Mean Fluorescence Intensity Ratios for Different Genotypes

| | | | | |
|---|---|---|---|---|
| | Locus specific | | | |
| Genotype | abnormality | Probe(s)* | MFI ± S.E. (n) | 95% C.I. |

(A) Comparison of MFI ratios with expected ratios from known genotypes

| | | | | |
|---|---|---|---|---|
| Biallelic | none | 16-1b or 16-2b | 1.01 ± 0.01 (16) | 1.00-1.03 |
| (non-deletion) | | HOXB1 | | |
| | none | TEKT3 or PMP22 | 1.01 ± 0.01 (4) | 0.97-1.05 |
| | | HOXB1 | | |
| Deletion | del (5′ ABL1) | 16-1b or 16-2b | 0.59 ± 0.02 (11) | 0.54-0.64 |
| | | HOXB1 | | |
| Triallelic | +9 or int dup(9) | 16-1b/HOXB1 | 1.37 ± 0.05 (7) | 1.25-1.50 |
| | int dup 17p12 | TEKT3 or PMP22 | 1.53 ± 0.04 (17) | 1.46-1.61 |
| | | HOXB1 | | |
| | int dup 17p12 | PMP22/16-1b | 1.48 ± 0.06 (3) | 1.21-1.75 |

(B) Identification of genotypes in blinded study of 20 control and 2 abnormal samples\hat{ }

| | | | | |
|---|---|---|---|---|
| Biallelic | none | TEKT3 | 1.00 ± 0.01 (60) | 1.00-1.01 |
| | | HOXB1 | | |
| Triallelic | CMT1A | TEKT3 | 1.51 ± 0.07 (6) | 1.33-1.70 |
| | | HOXB1 | | |

*Average of ratios of geometric mean fluorescence intensities for test: reference probes
\hat{ }Variance in MFI ratios among triplicate sets was 6.5 × 10⁻⁶ to 1 × 10⁻³

We claim:

1. A method of quantifying the copy number of a target nucleic acid sequence in a genome, said method comprising the steps of:
extracting nucleic acid containing said target nucleic acid sequence from a subject,
attaching a label to said target nucleic acid,
preparing a particulate-conjugated, low-copy, nucleic acid probe selected to complement said target nucleic acid sequence,
hybridizing said probe to said target nucleic acid to form a hybridization reaction product,
identifying said product through detection of said particulate, and
quantifying said copy number of said target nucleic acid sequence through detection of said label on said product.

2. The method of claim 1, wherein said particulate comprises a nanocrystalline particle.

3. The method of claim 1, wherein said particulate comprises a nanosphere.

4. The method of claim 1, wherein said particulate comprises a microsphere.

5. The method of claim 1, wherein said particulate comprises a spectrally-distinct polymer microsphere.

6. The method of claim 2, wherein said microsphere comprises polystyrene.

7. The method of claim 2, further comprising the step of binding a fluorochrome to a biological moiety on said microsphere surface.

8. The method of claim 2, further comprising a step selected from the group consisting of conjugating said probes to said microsphere via modified carbodiimide reaction wherein said microsphere has been carboxylated and conjugating said microsphere to said probe using an electrophilic tether, wherein said tether comprises N-chloroacetamidohexyl phosphoramidite.

9. The method of claim 1, wherein said particulate comprises an internally-dyed, fluorescent, polystyrene bead having a determined spectral address.

10. The method of claim 1, wherein said extracted target nucleic acid comprises DNA selected from the group consisting of genomic DNA and complementary DNA.

11. The method of claim 1, wherein said target nucleic acid is labeled by a process selected from the group consisting of labeling via nick translation with an identifying label, directly labeled during an in vitro nucleic acid replication reaction, end labeling, and random priming.

12. The method of claim 1, wherein said label is selected from the group consisting of fluorophores, enzymatic conjugates, fluorophore-tagged nucleotides, fluorescently-labeled antibodies bound to antigen-bearing nucleotides, biotin-dUTP, digoxygenin-dUTP, and combinations thereof.

13. The method of claim 1, wherein said low-copy probe has a copy number of 10 or less.

14. The method of claim 1, wherein the nucleic acid sequence of said probe is complementary to a single copy sequence in said subject's genome.

15. The method of claim 1, wherein a target nucleic acid containing a deletion of one or more base pairs exhibits a response ratio of about 0.1 to about 0.75 in comparison to a reference probe having a normal complement of said target nucleic acid sequence.

16. The method of claim 1, wherein a target nucleic acid containing an insertion or duplication of one or more base pairs exhibits a response ratio of about 1.25 to about 1.65 in comparison to a reference probe having a normal complement of said target nucleic acid sequence.

17. The method of claim 1, wherein multiple probes having distinct nucleic acid sequences conjugated to particulates having one or more distinct spectral addresses are hybridized to target nucleic acids.

18. The method of claim 1, wherein said particulate comprises a streptavidin-coated or carboxylated bead bound to a biotin moiety at the 3′ or 5′ terminus of said nucleic acid probe.

19. The method of claim 1, wherein the amount of target sequence detected per 1 μg of heterologous genomic nucleic acid sequence is within the range of 50 pg to 5 ng.

20. The method of claim 1, wherein differences in copy number are detected with a genomic resolution of as little as 60 bp.

21. The method of claim 1, said quantifying step including the step of detecting the spectral address of said product using flow cytometry.

22. The method of claim 1, said probe having a length of between 50 and 2500 bases.

23. The method of claim 1, said particulate having a diameter between 50 nm and 1 μm.

24. A method of detecting a suspected chromosomal abnormality in subject-derived genomic or complementary nucleic acid, said method comprising the steps of:
preparing a spectrally-encoded, fluorescent microsphere having a first spectral address,
identifying a target genomic nucleic acid probe sequence by ascertaining the nucleotide-by-nucleotide sequence of a target nucleic acid sequence wherein the abnormality is suspected to reside,
synthesizing a low copy target probe according to the identified target genomic nucleic acid probe sequence,
conjugating the target probe to a microsphere having a first spectral address,
synthesizing a reference probe selected to hybridize to a reference nucleic acid sequence having a known copy number of said target nucleic acid sequence,
conjugating the reference probe to a microsphere having a second spectral address,
reacting the target probe with a chromosomal target sequence containing the abnormality thereby causing the target probe to hybridize to the target sequence,
reacting the reference probe with a chromosomal reference sequence containing said chromosomal target sequence and thereby causing the reference probe to hybridize to the reference sequence,
detecting the hybridized target probe to ascertain the existence of the chromosome abnormality,
detecting the hybridized reference probe, and
quantifying the detected hybridized target probe by comparing the response of the detected hybridized target probe with the response of the detected hybridized reference probe response.

25. The method of claim 24, said abnormality being selected from the group consisting of differences in copy numbers of sequences within said genomic or complementary nucleic acid, duplications, deletions, inversions, transpositions, translocations, and combinations thereof.

26. The method of claim 24, said method detecting said abnormalities with a genomic resolution of as little as 60 bp.

27. The method of claim 24, said detecting steps using flow cytometry.

28. The method of claim 24, wherein said low-copy probe has a copy number of 10 or less.

29. A method of direct genomic quantitation of chromosomal abnormalities via flow cytometric detection of labeled target nucleic acid sequences hybridized to microsphere-conjugated low copy genomic or complementary nucleic acid probes, said method comprising the steps of:
preparing a spectrally-encoded, fluorescent microsphere having a diameter between 50 nm and 1 μm
synthesizing a low copy target probe selected to hybridize to a particular nucleic acid sequence in the target wherein the abnormality is suspected to reside
conjugating the target probe to the microsphere,
hybridizing the target probe to a target nucleic acid sequence,
detecting the hybridized target probe via flow cytometry, and
quantitating said abnormalities based on the results of said flow cytometry.

30. The method of claim 29, wherein the nucleic acid sequence of a probe is of a defined length between 50 and 2500 bases.

31. The method of claim 29, said abnormality being selected from the group consisting of differences in copy numbers of sequences within said genomic or complementary nucleic acid, duplications, deletions, inversions, transpositions, translocations, and combinations thereof.

32. The method of claim 29, said method detecting said abnormalities with a genomic resolution of as little as 60 bp.

33. A method of detecting chromosomal abnormalities, said method comprising the steps of:
preparing a hybridization probe by coupling a spectrally-encoded, polystyrene microsphere to a synthetic DNA sequence,
hybridizing said probe to genomic DNA,
detecting the product of said hybridization by flow cytometry.

34. The method of claim 33, said abnormality being selected from the group consisting of differences in copy numbers of sequences within said genomic or complementary nucleic acid, duplications, deletions, inversions, transpositions, translocations, and combinations thereof.

35. The method of claim 33, said method detecting said abnormalities with a genomic resolution of as little as 60 bp.

36. The method of claim 33, further comprising the step of comparing the results from said flow cytometry with flow cytometry results from a hybridization product having a known copy number corresponding to said abnormality.

37. The method of claim 33, wherein said probe is complementary to 10 or fewer locations in the genomic DNA.

38. The method of claim 33, said DNA being unamplified.


SEQUENCE LISTING (28 sequences)

SEQ ID NO: 1 - DNA, 2287 bp, Homo sapiens
aatagatgcaatgggggtgagagagaatgttcacttgccaagggaatgagggcagacaagagaaaggaaaattgagatgggaggaggtgacctgcagaggaattgaagagaaagaaattattgaggtaagagcaagcttatttctttctattttactttttcggggagaagatttgcagatgcttcatcctttcattttaagatgtcttccacagacgctaagaataacttgtctggaggaatctagggcttgtcttctaaccccaaatttgaacaaggcctgtatctgatctggcagccctgcttctgagagacttgagacttgttcaccttccaccttttcctcatcccggggagaatcttctcaccccatcctttgccttcataattaagggaacaatttgaggtctaaaggaagaagtgaatttcaaatcagtttaataaaacagcacaaatctgggaattgaggaatgcattccctttataacagctgaagtcaccaacaatctattattcatgacagcaagcaaagactgaggcaaaagtgccctaagttggctgggtgcagtcccagcactttggaaggccaaggcaagaggatcacttgagcccaggacttcgagaccagcctgggcaagatggtgagaccacatctctacaaaaataaataaatacaaaaattagctgggtgtggtggtgcctccctgaagtcccagctgctcagaagggaggatggcttgagcccaggagttcaaggctgcaatgagctatgaccacaccactgcactctagcctgggtgacagagaacctgtctctatttaaaaaaaaaaaaaaagtgcctcaagttatactgtcaattgcagtgattgcctcagctttccaaattcactatgctccccaattcatccctgagtcatgccaccttctcttgtgttagcagggacattaaaacttgcccaaattcctgctggctctcccagccccagcagaaaagactgggagaaccaagcaactttgctccctaggttcccaatctcctcctcctccctaaaagaagcttcgctgactggaataacctatggcccataggatttttggtggaccaaattaaactcaccagagagggtctgggtggacccaacaccaaaatgaaataaagttgatgtcctagtttgtatgatctgcttgtacataaaagagagcaaggagaacatagacatgtttggaactgaagttatctttgacagttttcactccttatcacactgagaatgcaggcaaggaaactatctaaccagaatggggatatattagaagtttaacctcagattctggtcctttctgtgaaggaagctgcttcagctgggggcttggcaggagcaatctaaattctctcattagaccttaaatgtctctatctgctctccccagcccaaatctagggaggccaaacccaagatcaaaggttggggctaggccagtgaggtggctcatgcctgtaatcccagcactttgggaggccaaggagggcagatcacctgaggtcgggagttcaagaccagcctgaccaacatggtgaaacccccatctctactaaaaaatacaaaaattagccgggtgtggtggcaggcacctataatcccagctactcaagaggctgaagcaggagaatcgcttgaacccgggaggcggaggttgcagtgagctgagatcgcaccattgcactccagcctgggcgacagagcaagactcaaaaaaaaaaaaaaaaagttggggcttaggaagtaaagaatattcataattttttccttctttctttctttttttttcaggcagggtttcgctctgttgccccaggctagagtgcaatggtgtaatcacggctccctacagctttgacctcctgggctcaagcgatcctcccacctcagcctcctgagtaggtaggactacaagcatgcaccaccacaagtttttcactatgagatgaattgtttttgttgctgttagagaaaggatatttcttttaatgcaagctaattaaactgtcataaatctggttgagaaccttagactgtatgtagtacatattgtaatgggattagtgctagctatgtgggccagaatagttgggttcaagcccttgcttggccattttttttttttttttttaatacagggtctcactttgtcacccaggctggagtgcagtggcgcaatctcggcccgctgcaacctctgcctcccaggcccaagcaatcctcccacctcaacctcccaagtagc

SEQ ID NO: 2 - DNA, 1344 bp, Homo sapiens
cggttctggaaccaaatcttgacctgtgtttcattgagctccagggtggcggcaatctccaccctccgggcccggctcaggtacttgttgaaatggaactccttttccagttctgtcagctgccttgtggtgaagttggtgcggaggccactgggcgagcccaggcctggctctgacaccttcgctaggggcggggcagggagaaaggccaggtcaattctctcacctctttctccttccgcttccctcctcccggggctggctctggacctccatttgccattctgcccaagtacagttgtcaaagttcccagggaccaccaggtacccccatggtgatcaccaagtctggagcagccttccccatacttccatcccccatgcacaccaggtcctgggtctgtgctgggtgcacagatcaggaagtggggtgtgtatggaaacttactcctggggtgaccggttacatactgggtggggagacctcacctgacctgagacgtaggttgtgctcttacctgtgtctaccagagccgctgaaagagaagaacccagcccagacccaggacatgtcactgcaggggaagcagagatgctttgggccccggagaaggggtggccacatctgagccctacctgtcttgggtgggtttctcttaaccttcatccagtcgaaggtccgggccgtgggggtgttaggttctgaagggcagggtgtttccttgtcctcggagaggagatcagcataggccggtgcaaagctcgcggtctgctcgttcccataagggggatgctgcggaggatatggccccggaccggctccacctgctccgtagccatcggacaagccccctagctgggccccgtagctcgagggatgaaaatagcctccgtctccttctgattgacccagagggtagtactgagaaggcccgtagctggggctgcaggcggcaggagcataccccgagggcgcggagctggggaagggcacccccagggtcgaaggcggctgctgggcgggatagccggagttctgctgaaacgcagggctggacagccccccaccgtagcggccctcgcttgcatagctgtcaaccgcctgagccgagcttgggggaaaggaggttggggcgctgtgggcgctgtaggcgctgggtccccggttacagagtgggtactctaagaaggagttcatcctattatagtccatgcgtcaaggccgcaggagggtcggcccgtttgggggagacaccctcttgccctacaacctttcggcagtatgtcacagcgctggggctcgaatccatggcctgacccgctcgcctgggcaagcgcttccct

SEQ ID NO: 3 - DNA, 94 bp, Homo sapiens
ttatcctctctctagccatccatagctgtagctggctcactcactcagacttaaataactccctatatctctctagagagccacaatgagcaca

SEQ ID NO: 4 - DNA, 2304 bp, Homo sapiens
gtggcttatgcctgtaatttcacactttgggaggccaaaggcaggtggattgcttgagcccaggagttcaagatcatcctgggcaacataagaagaccctgtctctacaacaagaacaaaagaaattagccagacgtgttatcacacctgtgttcccagctacttgagtggctgaggtgggagggtcacttgagctcgggaggccaatgctgcagtgagccatgatcacgccactgcactgcagcctgggtaacagagcaagaccccatctcaaaacaaagtttgagaactgttgctatggaaagtctatcaagaaggcacatagggatgtttgttgctgaagtactaacactgaatgatgtctgtgttgtggtgagtagaaaaaaagcaatttgaaaatcattatttatggtatggccccatttttgtaaaacaaaaatatctgtttatttgtgtatgtccaggaaaaaaaagtatggaaggattttacaccgaaatttttatctctagggacttggagctttaggtggaatcggagaaaactttctctttttggtttgtgtgatgttccaagtgtctttttacaacaaacttgggttatcttataatacgaaaaattcgaagatttaaaaactgaggccaggcacggtggctcatgcctataatcccagcactttgggaggttgacacatgtggatcacctgaggtcaggagtttgagaccagcttgaccaagaaggtaaaaccctgtctctactaaaaatacaaaaattagctgggcgtggttgcaggtgcctgtagtcccagctactcgggaggctgagacaggagagttgcttagacccaggaggtcgaggttgcaacgagctgagattgcacgccactgcactcccagcctgggcgacggagtgagactccatctcaaaataaataaataaataaaaataaaaactgaattgatgacagcccaacctgtcacttttttcagatccctttttatgaaagaatttgcttaagttgtgtctgaagacaaaccaatttctttggaccctgggtattctttttctaagggaataccatgttattttgtgttacagattgtttgcgatctttcataggctgatctttctagagttggttaatatccatgtaggttagattgaaaaacttgaattcagaaatgtacggtgttggagcagacatggatctggaagccaagaatagcgttggtgttgttgttgatggtgaatctgaaagagtgggcagatggcaactttttgccagaatcattcagggtcaaagggctagtcatatttacaagggaaaagaggaaaccatgttattaaatatttcatgcaccatatgttcctttcaaatttcagtggcttgactaagaaaattcacttttgggagagttggtacaacaattgaaagcacttagtagtcaactttttccttagtgtttccttcttccttcaacaaacatttattttattgagcacagactatgacaggtcgtgttgtagtcactgatgatatatttgtaaacaaaacaatccctgccctcctagaacttacattctagtgggaggagacacatcataaataagctaagtaaataaaatacagatatgttagtgatatgtgctgaggagaagaataacatagagaaggggacagggaatggggtaggaggtgtaattttagttagggtggctagagaggcctgactgagagggcggcatttgattaaagacccgaaggaggaagatgagcagcaggccctgttgaaatatgtgaaagaatttcaggccgagggaaccaggatgcagaaacctgaggcagagcttgctagtttcaaggagcagcctagaggccactgcaccagagcaaagagcaaaaggagagatgcaggaggtaaaatggagggtgggaagagaatggctgaaggagagggtggccttattggcccatttcaaggattttgactttgattcatatgatgtgggaaggccttggagaattttccgaggggactatactatctgatgtatgtttcagtatgaaatagaatctctctggctgctatgatctgaaggaggacaagggagcagggattccacgggggcattgattgcacagagccaggtaagagtgatgatgcttggaccaaagtggtggcactgggtggagggtcagaaccgacaagatttgctctcggattagatgtgggatatgggggtgtgagaggggccaagtgtgactctcaggttttcgaactgaataactggaagaatagagttgc

SEQ ID NO: 5 - DNA, 101 bp, Homo sapiens
gatctttctagagttggttaatatccatgtaggttagattgaaaaacttgaattcagaaatgtacggtgttggagcagacatggatctggaagccaagaat

SEQ ID NO: 6 - DNA, 1383 bp, Homo sapiens
atttggaaagattatatccatctacttaatgctagggattggattgtggcacgtggactgtgaaaatgcaaaattggaggtatgtactttattttagagacagggtcttgctttgtcacccaggctggagtgtagtggcacagtcatagctcactgcagccttgaactcctgggctccagtgatcctcttgccttagcctcccaaagcaatgggatcgtacaggtgagccattgcacctggccatgtatgtacattttttataagcattcagttgtttccaatagtctaatacattttgtatggccttcttatttttcatgaaaccttactacccattttaagaaatttgtaatatagaagaaaacagaagtttcaattgtggggtaagtaaagaaagatgatcaaagatgatttgaccaaaagagttttacttcatttaaatgaagcatgttgtcaaagaaaatgaaagtgggaaaaaatgtgaaatcattttgtatcagttcaggacagaaccgtgcttgtgcttcagatctccaagtgtgaacacactgatgtgttgtgaagtgtgttgcaaatagaagcattttttgaatgggagggagagggatgtgttaacatgatttagctgggaattaaacccataggaatgtgtcgggcatattttctgtgactagggggtaaaaagattaaaatggctactgaggatgggcttcagtaagaggaggtctcccgaaggccttctctgccctgtgcctctgcagttccctgactccttgctgtgttcgtccctgcctttttggtagttgtgcccctgatgtcactcccctgtgtgttcagatcctacctgctggccaggtgcagtggctcacacctgtaatcccagcactttgggaacccaaggtgggacggtcctctgagaccaggagtttgagagagtggcctggacaacatagcaacaccctattagtactaaaaataaaaaagttagctaggcataataatgtcctgtgcttgtctagctacttgggaggctgaggcagaaggattacttgagtccaggagttcgaggctgaagcaaactgtgatcatatcattgtactccagcctgtgtgataaagcaagaccctatcctttaaaagtaaaaaacaaacaaacaaacaaaaaacaaacctgcttaacctttcaacactctcttccagtcccccctcccttctgaaaccatattttacatctctggtcctcattaaattttctgtaacttctgcccttgtaactcttctgcaacacaaatgaatacataatcatatacttcctagtagtatttcctaatagttttatgtgagtttcatggaagagagtgttgaaacgtaggtaggtttgtt

SEQ ID NO: 7 - DNA, 102 bp, Homo sapiens
atttggaaagattatatccatctacttaatgctagggattggattgtggcacgtggactgtgaaaatgcaaaattggaggtatgtactttattttagagaca

SEQ ID NO: 8 - DNA, 99 bp, Homo sapiens
accaactttcatgccctaggtgaaacggaggacgtcttgggaggtagagagttgcccaaggtctgaatccatgggatctactaagaacaagacccagac

SEQ ID NO: 9 - DNA, 102 bp, Homo sapiens
agggcagctcatgacatgcctccatgttttccatgtgggtcatcaaacaacagagcaccagagagggttgaactaaatgaatactcaagtgccttccagtcc

SEQ ID NO: 10 - DNA, 81 bp, Homo sapiens
aaaggcgatctcctcctgctcctgctagagcgcagcgagcgcacggcgtttccgcagtaatcctgagcacggcaaggaccc

SEQ ID NO: 11 - DNA, 81 bp, Homo sapiens
attcataccagcaacagcagtggcatgatgcgggcaggattgccagtgtccgtgcatgcgtttgcactggtgattgcagtg

SEQ ID NO: 12 - DNA, 81 bp, Homo sapiens
gcaatagtggtggacaagttggcacctcgattgtctcagctcaaggttctgcacaaagtgtttagtactggagtagcagaa

SEQ ID NO: 13 - DNA, 81 bp, Homo sapiens
tacccaccaccctctaaccaccaaatggtctatcctagatggtgaagttgggttaattgttagacacggtggaatctttat

SEQ ID NO: 14 - DNA, 81 bp, Homo sapiens
cggcagctaggggcaggagtgcaagaatgatgtatcctttcaagtatcatgaccatcggggtgaattgctgagttctgggg

SEQ ID NO: 15 - DNA, 81 bp, Homo sapiens
taagtccttggcattacttaaagaaggccattagatatatgtcacgtgtgagagtgaggtttgcagctgggaagtaagcct

SEQ ID NO: 16 - DNA, 81 bp, Homo sapiens
gaaacagtcatcgagtgggatgacaacttggaaaggctttgtgggcctttgtgacctcatggtatgcatcccctcagggtg

SEQ ID NO: 17 - DNA, 81 bp, Homo sapiens
tggcgtctgctaggagtctcctatccggcgtttgtgccgcagaaaacctttaacctgcgttttacccgtaaacattttcct

SEQ ID NO: 18 - DNA, 81 bp, Homo sapiens
aaaagcataagttttagtcacatcaaaatataaacaatcttgaaattaattcataagtaaatcaatagatatcttgaaatt

SEQ ID NO: 19 - DNA, 81 bp, Homo sapiens
ttcggagagttacccaggtgtagtacccttccaagtagataatgattgaaagagtatgtgctggaggtgtatgatattttc

SEQ ID NO: 20 - DNA, 81 bp, Homo sapiens
ggttttgattttgatatactccagtttatctatttttgtgtttccttagcttttggtgttgtagtcaagaaatcattgcca

SEQ ID NO: 21 - DNA, 81 bp, Homo sapiens
tgcattctttttaaaaaatatttttagtctttacatttcagaatcagtatttcaaatgtgacttgtaggactacgtgcagt

SEQ ID NO: 22 - DNA, 81 bp, Homo sapiens
cttgctgttgtgccgttctgccccgatggtatcctgtccgctcgcattggggcgcgtcccccatccgcccccaactgtggt

SEQ ID NO: 23 - DNA, 81 bp, Homo sapiens
ggttactgattcataggggtatgtcgagggcttttgtctggaattccctgcttaccagtagatgattggtttacatatttt

SEQ ID NO: 24 - DNA, 81 bp, Homo sapiens
ggccgcagaggcaggctggcgcgcatgctcaggcggggatgtgtgcgaagcctgccgctgctgcagcgagtctggcgcaga

SEQ ID NO: 25 - DNA, 81 bp, Homo sapiens
agtatttcaaatgtgacttgtaggactacgtgcagtgaggaagattgttaatcatcctattctgaccaggagttctgacaa

SEQ ID NO: 26 - DNA, 81 bp, Homo sapiens
caaacactgggacccagaatagtaccctgtggtcatagtgcccacagttcactaagcaccctcacaggtctttgacagaac

SEQ ID NO: 27 - DNA, 81 bp, Homo sapiens
tttacctgcgcacttgcagatctttctccaggagtgagtttaaggtccgcacctccgctctggatggtgagcggagcacag

SEQ ID NO: 28 - DNA, 81 bp, Homo sapiens
aagacacttcaatcacccgcagcccatccatctgagagcgacatggaagcgtcaaaaacatctttcttcaaagacttttgg